(19) United States
(12) Patent Application Publication
(10) Pub. No.: US 2013/0259851 A1
(43) Pub. Date: Oct. 3, 2013

(54) USE OF PROKARYOTIC SPHINGOSINE-1-PHOSPHATE LYASES AND OF SPHINGOSINE-1-PHOSPHATE LYASES LACKING A TRANSMEMBRANE DOMAIN FOR TREATING HYPERPROLIFERATIVE AND OTHER DISEASES

(71) Applicants: Uwe Zangemeister-Wittke, Buchs (CH); Andrea Huwiler, Bern (CH); Markus G. Grutter, Hochwald (CH); Florence Bourquin, Biel (CH)

(72) Inventors: Uwe Zangemeister-Wittke, Buchs (CH); Andrea Huwiler, Bern (CH); Markus G. Grutter, Hochwald (CH); Florence Bourquin, Biel (CH)

(73) Assignees: UNIVERSITAT ZURICH, Zurich (CH); UNIVERSITAT BERN, Bern (CH)

(21) Appl. No.: 13/991,394

(22) PCT Filed: Nov. 30, 2011
(86) PCT No.: PCT/EP11/71446

§ 371 Date: Jun. 3, 2013

(30) Foreign Application Priority Data
Dec. 1, 2010 (EP) 10193347.1

(51) Int. Cl. A61K 38/51; A61K 31/7088

(52) U.S. Cl. CPC A61K 38/51; A61K 31/7088
USPC 424/94.5; 435/232; 435/252.33; 435/320.1; 514/44 R; 536/23.2

(57) ABSTRACT

The present invention relates to the use of prokaryotic sphingosine-1-phosphate lyases (S1PL) and S1PLs that lack a transmembrane domain or of a nucleic acid encoding such an S1PL in the prevention or treatment of a disease condition associated with elevated levels of sphingosine-1-phosphate (S1P), and for which S1P elevation is directly or indirectly causative. In addition, the invention relates to a new product in the form of S1PL lacking the N-terminal loop domain.

The present invention relates to the use of prokaryotic Sphingosine-1-phosphate lyases (S1PL) and S1PLs that lack a transmembrane domain or of nucleic acids encoding such S1PLs in the prevention or treatment of a disease condition associated with elevated levels of sphingosine-1-phosphate (S1P).

Sphingolipids are essential constituents of cellular membranes and serve as signalling molecules involved in various physiological and pathophysiological processes. Sphingosine-1-phosphate (S1P) plays a key role in regulating cell proliferation and survival, migration, angiogenesis, inflammatory processes and immune functions. S1P is present in blood at high nanomolar concentrations due to the S1P-producing activity of sphingosine kinases in various cell types including mast cells, erythrocytes and vascular endothelial cells. In blood S1P is bound to serum albumin and high density lipoproteins, which serve as buffers to decrease the pool of free S1P known to promote cardiovascular inflammation. Sphingosine-1-phosphate levels in plasma and HDL are altered in coronary artery disease. High levels of S1P are also generated by sphingosine kinases overexpressed in cancer cells, where S1P contributes to malignant progression and drug resistance as part of the sphingolipid rheostat counteracting pro-apoptotic sphingosine and ceramide. In addition to its intracellular function, upon secretion S1P may exacerbate disease progression by auto- and paracrine stimulation of S1P cell surface receptors. So far, five receptor subtypes have been identified and denoted as S1P₁₋₅. Their activation triggers downstream signaling via MAPK, P13K, cAMP and other mediators of cellular responses. Subsequent biological effects include cytoskeletal rearrangements, cell proliferation and migration, invasion, vascular development, platelet aggregation and lymphocyte trafficking.

Although elevated S1P is causal or at least contributory to major human diseases, its cytoprotective effect is also important to maintain the function of normal vital tissues such as the immune and the cardiovascular system. To sustain controlled amounts of this highly bioactive lipid in tissues, S1P is irreversibly degraded by intracellular S1P lyase. Decreasing the concentration of extracellular S1P or antagonizing S1P receptors may have therapeutic potential for various pathologic conditions including cancer, fibrosis, inflammation, autoimmune diseases, diabetic retinopathy and macular degeneration. The sphingosine analogue FTY720 (fingolimod) is a clinically advanced immunosuppressive agent used for the treatment of autoimmune diseases. Upon phosphorylation in vivo FTY720 acts as an agonist on all S1P receptors, except for S1P₂. On the other hand, FTY720-phosphate may also indirectly antagonize S1P receptor signaling by receptor downregulation, thereby rendering cells unresponsive to S1P. This ambivalent behaviour may result in unpredictable effects in vivo limiting the therapeutic use of this compound. As a more predictable approach, an anti-S1P antibody has recently been described, which acts as a molecular sponge to reduce the pool of endogenous circulating S1P. However, it is questionable whether the reversible absorption of S1P with a neutralizing antibody can compete with the continuous release of S1P from blood and various other cell types.

S1P lyase has been cloned from various species including yeast (Saba et al. (1997) J Biol Chem 272(42): 26087-26090), mouse (Zhou et al. (1998) Biochem Biophys Res Commun 242(3): 502-507) and human (Van Veldhoven et al. (2000) Biochim Biophys Acta 1487(2-3): 128-134); see also sequences of S1P lysases disclosed in WO-A-99/16888 and WO-A-99/38983. Recently the structure and function of S1P lyase from Symbiobacterium thermophilum (StSPL) has been cloned and charcterised (Bourquin et al. (2010) Structure 18(8): 1054-1065). In contrast to the enzymes from yeast, mouse, human and other species, StSPL lacks a typical transmembrane sequence (Bourquin et al. (2010), supra), and its structure solved at 2.0 Å resolution revealed that the active protein is a typical type I-fold dimeric pyridoxal-5′-phosphate (PLP)-dependent enzyme in which residues from both subunits contribute to the active sites.

The technical problem underlying the present invention is to provide a novel therapeutic regimen for diseases associated with elevated levels of S1P, and for which S1P elevation is directly or indirectly causative.

The solution to the above technical problem is provided by the embodiments of the present invention as described herein and characterised in the claims.

In particular, the present invention is based on the finding that certain isolated S1P lyases that—in comparison to typical S1P lyases from yeast, mouse, human other higher organisms—lack a transmembrane domain are functional enzymes in an extracellular context in vitro and in vivo.

Furthermore, the inventors found out that proklaryotic S1P lyases in general (i.e. also prokaryotic S1P lyases having a transmembrane domain—in contrast to most enzymes having a transmembrane domain from eukaryotic species—can be successfully expressed in expression systems and are also functional enzymes in an extracellular context.

Thus, according to a first aspect, the present invention generally provides the use of a sphingosine-1-phosphate lyase (S1PL) lacking a transmembrane domain (i.e. a transmembrane domain-free S1PL) for preventing or treating a pathologic condition associated with elevated levels of sphingosine-1-phosphate.

According to a second aspect, the present invention relates to the use of a prokaryotic S1PL, in particular a prokaryotic S1PL containing a transmembrane domain, for preventing or treating a pathologic condition associated with elevated levels of sphingosine-1-phosphate.

The present invention also contemplates the use of functional derivatives or mutants of a prokaryotic or of a transmembrane domain-free S1PL for the treatment or prevention of the pathologic conditions as disclosed herein. Further subject matter of the present invention relates to the use of a nucleic acid encoding a prokaryotic or a transmembrane domain-free S1PL or a functional derivatives or mutants thereof, in particular for expression of such a prokaryotic or a transmembrane domain-free S1PL or functional derivatives or mutants thereof, for the indications as described herein.

Such use may be realised by using the above-mentioned proteins or nucleic acids for the manufacture of a medicament for the treatment or prevention of the indications as described herein.

The present invention further discloses the general use of a prokaryotic or of a transmembrane domain-free S1PL or functional derivatives or mutants thereof or a nucleic acid coding for a prokaryotic or a transmembrane domain-free S1PL or functional derivatives or mutants thereof as a medicament per se.

Pathologic conditions associated with elevated levels of S1P include hyperproliferative diseases, inflammation, autoimmune diseases, diabetic retinopathy and macular degeneration. Hyperproliferative diseases treatable (and preventable) according to the invention comprise cancer, fibrosis and aberrant angiogenesis.

The term “transmembrane domain-free S1PL” relates to isolated polypeptides showing the structural features of typical type I-fold dimeric pyridoxal-5′-phosphate (PLP)-dependent enzymes capable of degrading S1P but lacking a transmembrane sequence (typically a transmembrane helix). Such proteins may be obtained directly from naturally occurring sequences or may be as well derived from S1PL enzymes that naturally have a transmembrane domain (such as the sequences of S1PLs from yeast, mouse, human and other organisms published as mentioned above) by eliminating the transmembrane domain, e.g. eliminating the transmembrane domain by genetically engineering a corresponding deletion mutant of the transmembrane domain-containing wild-type. The transmembrane domain to be eliminated from a given lyase may be detected in a given sequence using publically or commercially available structure prediction tools; see, for example, SOSUI (Hirokawa et al. (1998) Bioinformatics Vol. 14 S. 378-379) and TMpred (Hoffmann et al. (1993) Biol. Chem. Hoppe-Seyler 374, 166).

A “functional derivative” of an S1PL useful in the context of the present invention is a polypeptide showing the activity of an S1PL which has been chemically altered compared to the wild-type protein. For example, a derivative may be a functional fragment of the wild-type sequence. Other derivatives contemplated according to the present invention have specific functional groups or smaller or larger chemical moieties added to the polypeptide. As specifically preferred examples, polyethylene glycol (PEG) or albumin-conjugated or labelled derivatives of a prokaryotic or a transmembrane domain-free S1PL may be mentioned. Preferred labels according to the present invention are for example fluorophors, prosthetic groups, such as biotin, or radiolabels.

A “mutant” or “variant” of a S1PL of use according to the present invention may be derived from a wild-type polypeptide by addition, deletion and/or substitution of one or more amino acids such that the mutant or variant has an altered sequence compared to the wild-type amino acid sequence. Functional mutants typically have 95%, 96%, 97%, 98% or 99% or even higher sequence identity to the wild-type sequence. However, functional mutants may also be obtained in case of, e.g. amino acid substitutions, if up to 25% of the wild-type amino acid positions are substituted. Such amino acid substitutions are preferably conservative. A conservative substitution is one in which an amino acid is substituted for another amino acid that has similar properties, such that a person skilled in the art of protein chemistry would expect the secondary structure and hydropathic nature of the resulting polypeptide to be substantially unchanged in comparison to the native polypeptide. As a rule, the following amino acids represent conservative substitutions: (i) Ala, Pro, Gly, Glu, Asp, Gln, Asn, Ser, Thr; (ii) Cys, Ser, Tyr, Thr; (iii) Val, Ile, Leu, Met, Ala, Phe; (iv) Lys, Arg, His; (v) Phe Tyr, Trp, His. Substitutions, deletions and/or amino acid additions may occur at any position in the sequence provided that the polypeptide retains the activity an S1P lyase.

Especially useful mutants in the context of the present invention include S1PLs as defined herein having one ore more mutations of specific residues undergoing regulation by nitrosylation or phosphorylation (i.e. Tyr, Ser, Thr)—known to occur in the human enzyme (see Zhan & Desiderio (2006) Analytical Biochemistry 354 (2006) 279-289). Replacing conserved Tyr, Ser and Thr close to the active site by, for example, Phe or Ala can prevent the down-regulation that may target the native enzyme. Additional amino acid sequences (such as linkers, tags and/or ligands) are preferably present at the amino terminus and/or the carboxy terminus. Such additional sequences may be useful, e.g., to facilitate purification or detection or to improve extracellular stability of the polypeptide. Examples of (poly)peptide tags to facilitate purification are GST, GB1 and His-tags.

The polypeptides useful in the present invention may be prepared using any of a variety of techniques well known in the art. Preferred is a recombinant expression of a S1PL as disclosed herein in a suitable host. Corresponding techniques are well known, see, for example, the latest edition of Ausubel et al. (ed.) Current Protocols in Molecular Biology, Wiley; N.Y., USA.

Preferred transmembrane domain-free S1P lyases of use according to the present invention are from prokaryotes such as bacteria. Particularly preferred representatives include corresponding bacterial S1PL proteins from the genera Symbiobacterium, Erythrobacter, Myxococcus, Burkhodaria, Streptomyces, Stigmatella, Rhodococcus, Plesiocystis and Fluoribacter. More preferably the S1PL lacking a transmembrane domain for use according to the invention is derived from Symbiobacterium thermophilum, Erythrobacter fitorafis (preferably strain HTCC2594), Myxococcus xanthus (preferably strain DK 1622), Burkholderia thailandensis (preferably strain E264), Burkholderia pseudomallei (preferably strain 1106a, 305, Pasteur 52237, S13, 406e, 1655 or MSHR346), Erythrobacter sp. (preferably strain NAP1), Myxococcus fulvus (preferably strain HW-1), Streptomyces sp. (preferably strain e14), Stigmatella aurantiaca (preferably strain DW4/3-1), Rhodococcus erythropolis (preferably strain SK121), Plesiocystis pacifica (preferably strain SIR-1) or Fluoribacter dumoffii. Especially preferred examples of useful prokaryotic S1PLs lacking a transmembrane domain are summarized in the following Table 1:

TABLE 1
Preferred examples of prokaryotic S1PLs
lacking a transmembrane domain

| | UNIPROT | Length | SEQ |
|---|---|---|---|
| | accession | (amino acid | ID |
| Organism | # | residues) | NO: |
| Symbiobacterium thermophilum | Q67PY4 | 507 | 1 |
| Erythrobacter litoralis (strain | Q2NDU7 | 412 | 2 |
| HTCC2594) | | | |
| Myxococcus xanthus (strain DK | Q1D8D2 | 509 | 3 |
| 1622) | | | |
| Burkholderia thailandensis | Q2T8I9 | 473 | 4 |
| (strain E264/ATCC 700388/ | | | |
| DSM 13276/CIP 106301) | | | |
| Burkholderia thailandensis | Q2T8I7 | 473 | 5 |
| (strain E264/ATCC 700388/ | | | |
| DSM 13276/CIP 106301) | | | |
| Burkholderia pseudomallei (strain | A3P8X5 | 473 | 6 |
| 1106a) | | | |
| Burkholderia pseudomallei (strain | A3P8Y1 | 498 | 7 |
| 1106a) | | | |
| Burkholderia pseudomallei 305 | A4LG68 | 473 | 8 |
| Burkholderia pseudomallei Pasteur | A8KE32 | 485 | 9 |
| 52237 | | | |
| Burkholderia pseudomallei Pasteur | A8KE38 | 473 | 10 |
| 52237 | | | |
| Burkholderia pseudomallei S13 | B1H9E3 | 485 | 11 |
| Burkholderia pseudomallei 406e | A8EMX3 | 473 | 12 |
| Burkholderia pseudomallei 1655 | B2HAT2 | 485 | 13 |
| Burkholderia pseudomallei 406e | A8EMW8 | 485 | 14 |
| Burkholderia pseudomallei 305 | A4LG74 | 507 | 15 |
| Burkholderia pseudomallei S13 | B1H9D7 | 493 | 16 |
| Burkholderia pseudomallei 1655 | B2HAT7 | 493 | 17 |
| Burkholderia pseudomallei MSHR346 | C4I9Q9 | 493 | 18 |
| Erythrobacter sp. NAP1 | A3W9L7 | 412 | 19 |
| Myxococcus fulvus HW-1 | D6N158 | 509 | 20 |
| Streptomyces sp. e14 | D6KE63 | 503 | 21 |
| Stigmatella aurantiaca DW4/3-1 | Q08TY4 | 440 | 22 |
| Stigmatella aurantiaca DW4/3-1 | Q08VE4 | 506 | 23 |
| Rhodococcus erythropolis SK121 | C3JKZ9 | 518 | 24 |
| Plesiocystis pacifica SIR-1 | A6GEB7 | 509 | 25 |
| Fluoribacter dumoffii | C6ZD42 | 597 | 26 |
| Symbiobacterium thermophilum | Q67PY4 | 450 | 36 |

Specific sequences include proteins comprising, more preferably consisting of, the amino acid sequences according to SEQ ID NO: 1 to 26 and 36. With respect to functional mutants (or variants) and derivatives thereof, it is referred to the above description.

Further preferred transmembrane-free 51 P lyases useful in the context of the present invention are from amoeba such as Polysphondylium pallidum, more preferably strain PN500. A specific example of transmembrane domain-free S1PL from this organism is a protein having (or comprising) an amino acid sequence according to SEQ ID NO: 27.

Especially preferred S1P lyases in the context of the present invention are from Symbiobacterium thermophilum and include the protein of SEQ ID NO: 1 and SEQ ID: 36 as well as functional derivatives or mutants thereof as defined above. Typical examples of variants of the proteins in the context of the present invention, in particular proteins of SEQ ID NO: 1 and 36 include His-tagged versions of the polypeptide such as the sequences of SEQ ID NO: 28, 37 and 38. A highly preferred polynucleotide sequence encoding the protein of SEQ ID NO: 1 is shown in SEQ ID NO: 29. A polynucleotide encoding the protein of SEQ ID NO: 28 is shown in SEQ ID NO: 30. These sequences represent especially preferred nucleic acids for use according to the invention.

Typical examples of variants of the protein of SEQ ID: 36 include His-tagged versions of the polypeptide such as the sequence of SEQ ID NO: 37. A highly preferred variant the protein of SEQ ID NO: 36 is shown in SEQ ID NO: 38.

Preferred prokaryotic S1PLs containing a transmembrane domain include corresponding S1PL proteins from Legionella, in particular Legionela pneumophila (preferably strain Paris, Philadelphia or Lens) and Legionella jamestowniensis, as well as from marine proteobacteria such as the marine gamma proteobacterium HTCC2143. Especially preferred examples of useful prokaryotic S1PLs containing a transmembrane domain are summarized in the following Table 2:

TABLE 2
Preferred examples of prokaryotic S1PLs
containing a transmembrane domain

| | UNIPROT | Length | SEQ |
|---|---|---|---|
| | accession | (amino acid | ID |
| Organism | # | residues) | NO: |
| Legionella pneumophila (strain Paris) | Q5X3A8 | 605 | 31 |
| Legionella pneumophila subsp. | Q5ZTI6 | 601 | 32 |
| pneumophila (strain Philadelphia 1/ | | | |
| ATCC 33152/DSM 7513) | | | |
| Legionella pneumophila (strain Lens) | Q5WUR6 | 605 | 33 |
| Legionella jamestowniensis | C6ZD45 | 601 | 34 |
| Marine gamma proteobacterium | A0YDC8 | 410 | 35 |
| HTCC2143 | | | |

Specific sequences include proteins comprising, more preferably consisting of, the amino acid sequences according to SEQ ID NO: 31 to 35. With respect to functional mutants (or variants) and derivatives thereof, it is referred to the above description.

As already outlined above, for reducing elevated levels of S1P according to the invention, it is also contemplated to use nucleic acids coding for a S1PL (or functional mutant or derivative thereof) as defined above. Typically, such nucleic acids are prepared for the expression of the S1PL. Thus, the term “nucleic acid encoding a transmembrane domain-free S1PL or functional derivative or mutant thereof” or “nucleic acid encoding a prokaryotic S1PL” includes corresponding vectors into which the respective polynucleotide has been inserted. The vector preferably includes one or more vector elements known in the art such as origin of replication, selectable marker(s), promoter(s), enhancer(s), polyadenylation signal(s) etc. Preferably, the nucleic acid, most preferably in the form of a corresponding vector for expression of the S1PL (for example, a prokaryotic S1PL) as defined herein, is introduced into a cell of the patient to be treated.

Further subject matter of the present invention is a method for the prevention or treatment of a pathologic condition associated with elevated levels of sphingosine-1-phosphate (S1P) comprising the step of administering to a patient in need thereof a therapeutically effective amount of a prokaryotic or a transmembrane domain-free sphingosine-1-phosphate lyase (S1PL) or functional derivative or mutant thereof as described herein or a nucleic acid encoding a prokaryotic or a transmembrane domain-free S1PL or a functional derivative or mutant thereof as disclosed herein.

The S1PL or nucleic acid useful in the context of the present invention is typically present in a pharmaceutical composition, usually in combination with a pharmaceutically acceptable carrier and optionally adjuvants. The pharmaceutical compositions comprise from approximately 1% to approximately 99.9% active ingredient.

The administration of the active substance, in particular the S1PL or mutant or derivative thereof, may be carried out by any method known to those in the art suitable for delivery to the human organism. Preferably, the S1PL useful in the context of the present invention is administered by intravenous injection or intraarterial injection. In some aspects, administering comprises transdermal, intraperitoneal, subcutaneous, sustained release, controlled release or delayed release administration of the prokaryotic or the transmembrane-free S1PL (or functional derivative or mutant thereof).

For parenteral administration, preference is given to the use of solutions of the polypeptide, and also suspensions or dispersions, especially isotonic aqueous solutions, dispersions or suspensions which, for example, can be formed shortly before use. The pharmaceutical compositions may be sterilized and/or may comprise excipients, for example preservatives, stabilizers, wetting agents and/or emulsifiers, solubilizers, viscosity-increasing agents, salts for regulating osmotic pressure and/or buffers and are prepared in a manner known per se, for example by means of conventional dissolving and lyophilizing processes.

The dosage of the active ingredient depends on the species, its age, weight, and individual condition, the individual pharmacokinetic data, and the mode of administration.

The selection of appropriate additives and formulation of pharmaceutical preparations for use in the present invention is known to the skilled artisan and guidelines can be found in the latest edition of Remington's Pharmaceutical Sciences (Maack Publishing Co. Easton, Pa., USA).

The “patient” according to the present invention is a human or an animal, in particular mammals such as production animals, e.g. cattle, sheep, pig etc.

Further subject matter of the present invention is a deletion mutant of a transmembrane domain-free Spingosine-1-phosphate lyase (S1PL) which, in comparison to the respective wild-type species, lacks the N-terminal loop domain. The “N-terminal loop domain” according to the present invention is the N-terminal part of the protein. This part, especially in the case of the S1PL of Symbiobacterium thermophilum (StSPL), is not visible on the electron density map during analysis of the crystal structure of the protein using X-ray diffraction. The N-terminal loop domain of StSPL is denoted as Nt-FLEX domain. Therefore, a deletion variant of StSPL lacking the N-terminal loop domain is denoted as ΔNt-FLEX variant. The invention is also directed to functional derivatives or mutants of such a deleted S1PL and to a nucleotide sequence encoding a S1PL which lacks an N-terminal loop or a functional derivative or mutant thereof.

Typical S1PL lacking an N-terminal loop domain according to the present invention are derived from a bacterium selected from the group consisting of Symbiobacterium thermophilum, Erythrobacter litoralis, Myxococcus xanthus, Burkholderia thailandensis, Burkholderia pseudomallei, Erythrobacter sp., Myxococcus fulvus, Streptomyces sp., Stigmatella aurantiaca, Rhodococcus elythropolls, Plesiocystis pacifica and Fluoribacter dumoffii, more preferably from bacteria such as Symbiobacterium thermophilum and include the protein of SEQ ID NO: 36 as well as functional derivatives or mutants thereof as defined above.

It is further preferred that the S1PL lacking an N-terminal loop domain has an amino acid sequence which lacks 50 to 60 amino acids of the wild-type sequence at its N-terminus, more preferred 55 to 58 amino acids, especially preferred 57 amino acids.

The invention is also directed to polynucleotides encoding such S1PL deletion mutants.

The protein of SEQ ID NO: 36 is a mutant of the wild-type S1PL of Symbiobacterium thermophilum which was constructed by deleting 57 amino acids at the N-terminus of the wild-type protein (SEQ ID NO: 1).

Typical examples of variants of the protein of SEQ ID NO: 36 include His-tagged versions of the polypeptide such as the sequence of SEQ ID NO: 37. Other tags known by the person skilled in the art, as for example HA-tags, Myc-tags or maltose-binding-protein-tags can also be used to produce variants of the protein of SEQ ID NO: 36. A highly preferred variant the protein of SEQ ID NO: 36 is shown in SEQ ID NO: 38.

It is known that functional Spingosine-1-phosphate lyases as described herein, in particular StSPL, are usually dimers of two identical subunit proteins; see, for example Bourquin et al. (2010), supra. The person skilled in the art therefore is aware that the present invention is also directed to such dimers and uses thereof of the protein as described and claimed herein.

Further subject matter of the present invention is a vector containing a polynucleotide encoding an S1PL deletion mutant according to the present invention. Suitable vectors are, for example viruses or cloning vectors known to the person skilled in the art. It is further preferred that the vector enables expression of the S1PL deletion mutant.

Furthermore, the present invention provides a cell transformed with a polynucleotide encoding a deletion mutant of a transmembrane domain-free S1PL as described above and/or a vector containing such a polynucleotide. Suitable host cells according to the invention are, for example prokaryotic or eukaryotic cells. Host cells used in the context of the invention are prokaryotic cells, more preferred bacteria, especially preferred Escherichia coli-cells, and eukaryotic cells, for example yeast, insect or mammalian cells.

The present invention also discloses a method for the production of a deletion mutant of a transmembrane-free S1PL as characterised above comprising the steps of
(a) culturing a cell transformed with a polynucleotide or vector encoding an S1PL deletion mutant lacking the N-terminal loop domain as defined above in a culture medium under conditions allowing the expression of the protein; and
(b) purifying the protein from the cells and/or the culture medium.

This aspect of the present invention is based on the finding that certain isolated transmembrane-free S1PL which lack the N-terminal loop domain are functional enzymes in an extracellular context in vitro and in vivo.

Furthermore, the inventors found out that proteins according to SEQ ID NO: 36, 37 or 38 show higher recombinant expression yields in E. coli than wild-type S1PL, as for example wild-type StSPL. Furthermore, these proteins according to the present invention are easier to purify due to the lack of formation of a higher oligomeric state as observed for the wild-type protein.

Further subject matter of the present invention is the use of an S1PL lacking an N-terminal loop domain as defined herein as a medicament, in particular its use for the prevention or treatment of a pathologic condition associated with elevated levels of sphingosine-1-phosphate.

The present invention also discloses a pharmaceutical composition comprising a deletion mutant of a transmembrane-domain free S1PL as characterised above and/or a vector containing a polynucleotide encoding an S1PL deletion mutant according to the present invention and/or cells transformed with a polynucleotide as described above and/or a vector containing such a polynucleotide in combination with at least one pharmaceutically acceptable carrier, excipient and/or diluent. A suitable pharmaceutically acceptable carrier is for example water or an isotonic saline solution. These and other carriers as well as suitable excipients and diluents are known to the person skilled in the art and are for example set out in the latest edition of Remington's Pharmaceutical Sciences (Mack Publishing Co., Easton, Pa., USA).

Furthermore, the present invention provides a method for the treatment of a disease as mentioned above, preferably a pathologic condition associated with elevated levels of sphingosine-1-phosphate, comprising administering an effective amount of the pharmaceutical composition of the invention to a preferably mammalian, particularly human, patient in need of such treatment.

The figures show:

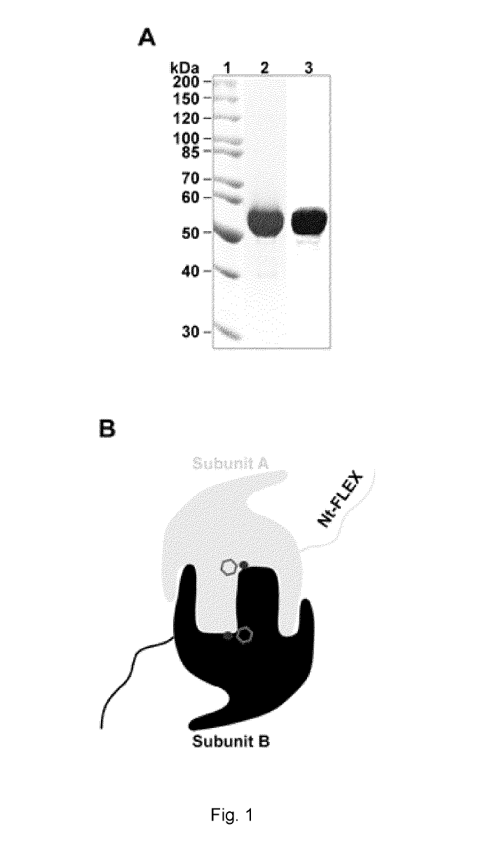

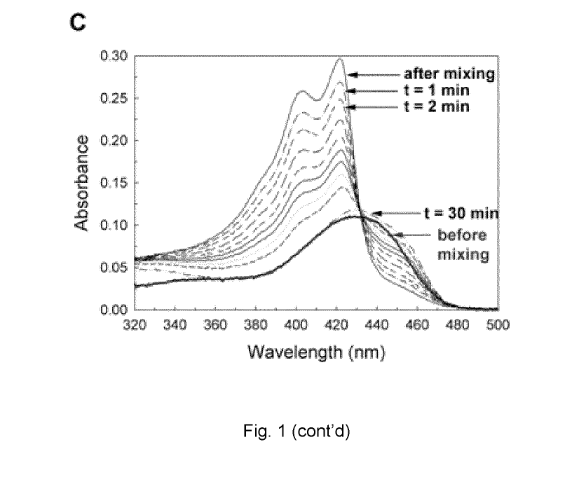

FIG. 1: Biochemical characterisation of StSPL. (A) Purity of the purified wild-type StSPL. The molecular weight marker is shown in lane 1, the pooled fractions after size-exclusion chromatography were detected by Coomassie staining of the gel (lane 2) and by Western blotting with an antibody recognizing the C-terminal His-tag (lane 3). (B) Schematic representation of the StSPL dimer. Subunit A is depicted in grey, whereas subunit B is in black. A phosphate ion found in the active site of both subunits is depicted as a dot, while the cofactor (PLP) is denoted by a hexagon. (C) Spectrophotometric activity assay of wild-type StSPL. The curve represents the visible spectrum of the native protein before the addition of substrate, corrected by the dilution factor. The black curves depict the visible spectra at regular intervals (1 min, 2, 4, 6, 8, 10, 12, 15, and 30 min) after addition of S1P. The transient peaks at 420 and 403 nm appearing upon addition of substrate correlate with protein activity. (D) Mass spectrometric activity assay of wild-type StSPL. The left panel depicts the reaction mixture measured just after mixing protein and substrate. The 163.07 and 380.26 kDa peaks correspond to the end product phosphoethanolamine and the substrate S1P, respectively. The right panel shows the reaction mixture after 75 min incubation at 20° C. No peak corresponding to S1P was detectable above background level.

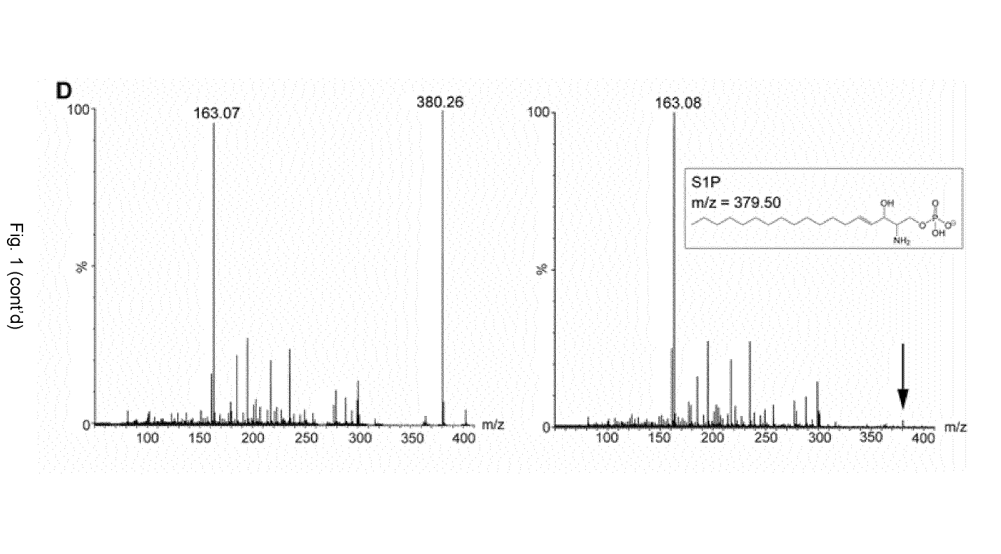

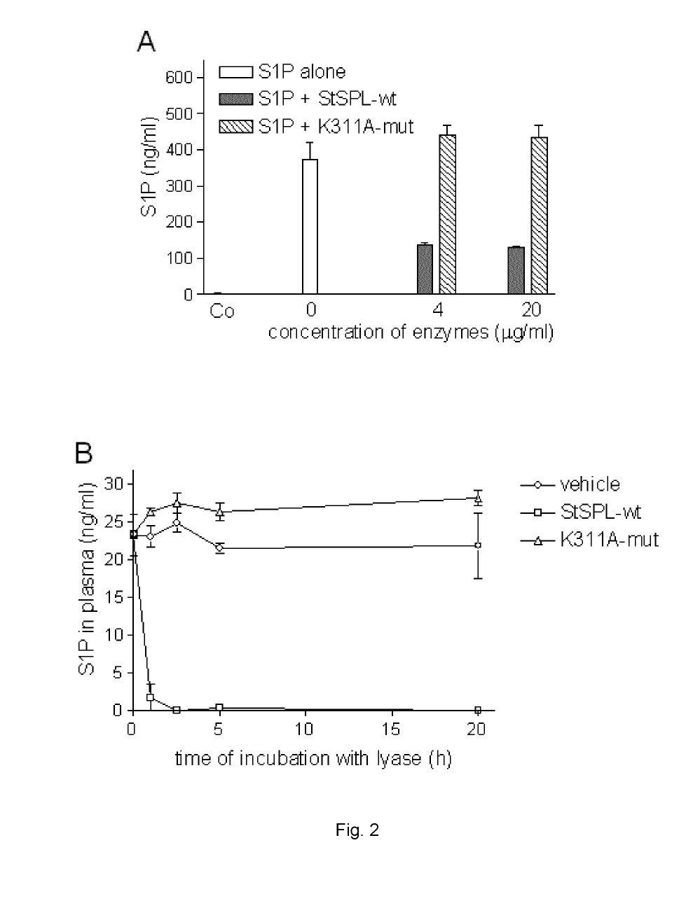

FIG. 2: Wild-type StSPL degrades S1P in vitro. (A) Medium (DMEM) was incubated for 30 min at 37° C. with either vehicle (Co) or S1P in the absence (0, open bar) or presence of the indicated concentrations of wild-type StSPL (StSPL-wt; closed bars) or the K311A mutant (K311A-mut; hatched bars). Thereafter, 100 μl of the medium was taken for lipid extraction and S1P was quantified by LC-MS/MS. Data are expressed as ng/ml of S1P and are means±SD (n=3). (B) Human plasma was incubated at 37° C. for the indicated time periods (in hours) with either buffer (vehicle, circles), 20 μg/ml of wild-type StSPL (StSPL-wt; squares), or 20 μg/ml of the K311A mutant (K311A-mut; triangles). 100 μl plasma was taken for lipid extraction and S1P was quantified by LC-MS/MS. Data are expressed as ng/ml of S1P and are means±SD (n=3).

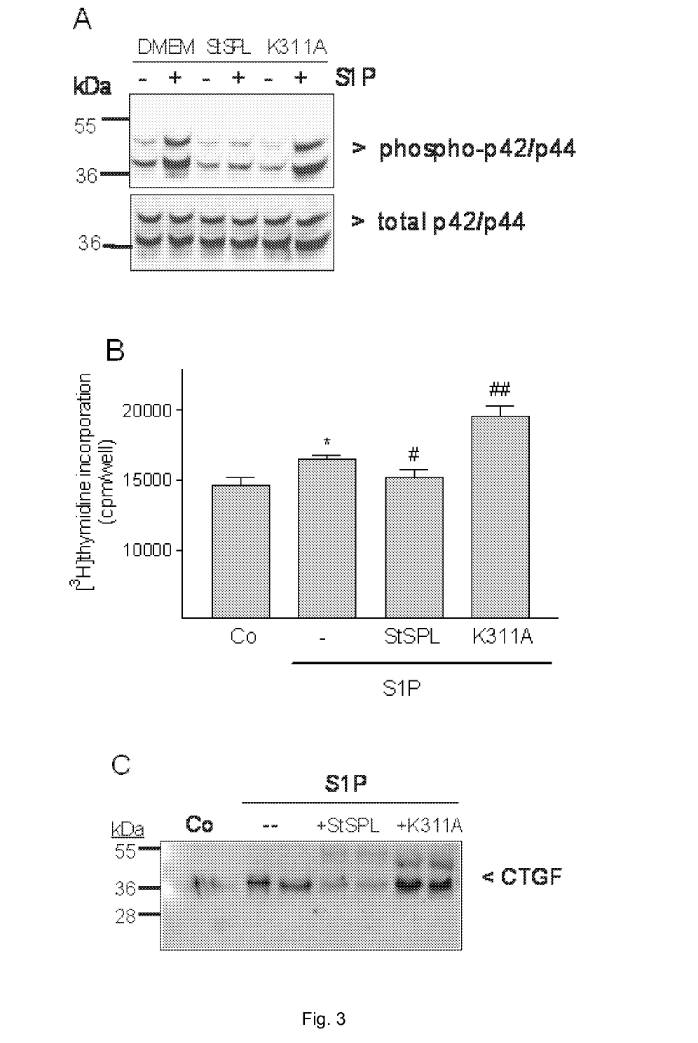

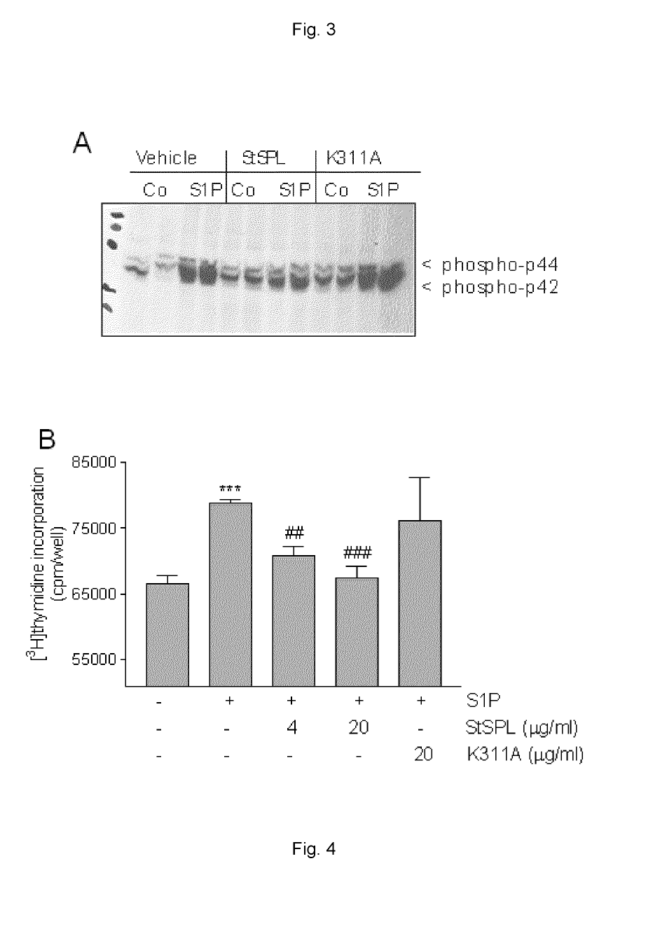

FIG. 3 Effect of StSPL on S1P-stimulated MAPK phosphorylation, cell proliferation and CTGF expression in renal mesangial cells. (A) Quiescent rat mesangial cells were treated for 10 min with either vehicle (DMEM, −) or S1P (1 μM) in the absence or presence of wild-type StSPL (10 μg/ml) or the K311A mutant (10 μg/ml). Thereafter, cell lysates were separated by SDS-PAGE, transferred to nitrocellulose and subjected to Western blotting using antibodies against phospho-p42/p44 (dilution of 1:1000, upper panel) and total p42/p44-MAPK (dilution each 1:6000, lower panel). Blots were stained by the ECL method according to the manufacturer's recommendation. Data are representative of five independent experiments. (B) Quiescent cells were treated for 28 h with either vehicle (Co) or S1P (1 μM) which had been pretreated for 30 min at 37° C. with either vehicle (−), wild-type StSPL (denoted StSPL, 20 μg/ml) or the K311A mutant (denoted K311A, 20 μg/ml) in the presence of [³H]thymidine. Incorporated radioactivity was measured as described in the Methods Section. Results are expressed as cpm/well of incorporated [³H]thymidine and are means±S.D. (n=4). (C) Quiescent cells were treated for 2 h as indicated above, and proteins were precipitated from the supernatants and taken for SDS-PAGE, transfer to nitrocellulose membranes and Western blotting using a CTGF-specific antibody (dilution 1:1000). *p<0.05 considered statistically significant when compared to the vehicle treated control values; #p<0.05, ##p<0.01 when compared to the S1P-treated values.

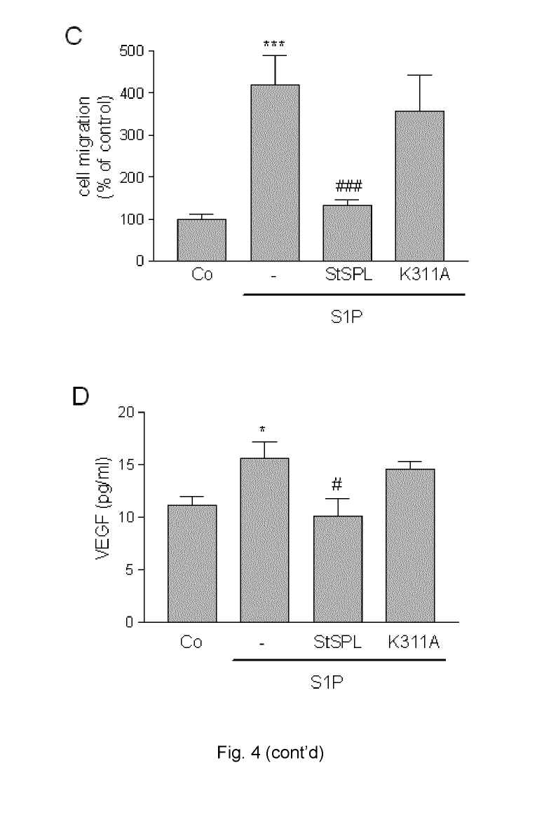

FIG. 4: Effect of StSPL on S1P-stimulated MAPK phosphorylation, cell proliferation, migration and VEGF production of endothelial cells. (A) Quiescent EA.hy 926 human endothelial cells were treated for 10 min with either vehicle (Co) or S1P (1 μM) in the absence or presence of wild-type StSPL (denoted StSPL, 10 μg/ml) or the K311A mutant (denoted K311A, 10 μg/ml). Cell lysates were prepared and separated by SDS-PAGE, transferred to nitrocellulose and subjected to Western blotting using antibodies against phospho-p42/p44 (dilution of 1:1000, upper panel) and total p42/p44-MAPK (dilution each 1:6000, lower panel). Data are representative of four independent experiments. (B) Quiescent cells were treated for 28 h with either vehicle (−) or S1P (1 μM), which had been pretreated for 30 min at 37° C. with either vehicle (−), wild-type StSPL or the K311A mutant, in the presence of [³H]thymidine. Incorporated radioactivity was measured as described in the Methods Section. Results are expressed as cpm/well of incorporated [³H]thymidine and are means±S.D. (n=4). (C) Quiescent cells were treated for 14 h with DMEM (Co) or S1P (1 μM) which had been pretreated for 30 min at 37° C. with either vehicle (−), wild-type StSPL or the K311A mutant. Thereafter, migrated cells were analysed as described in the Methods Section. Results are expressed as migrated cells per counted field and are means±S.D. (n=3). (D) Quiescent cells were treated for 48 h with either DMEM (Co) or S1P (1 μM), which had been pre-treated for 30 min with either vehicle (−), wild-type StSPL or the K311A mutant. Supernatants were taken for a VEGF-ELISA. Results are expressed as pg/ml of VEGF and are means S.D. (n=4). ***p<0.001 considered statistically significant when compared to the vehicle treated control values; #p<0.05, ##p<0.01, ###p<0.001 when compared to the S1P-treated values.

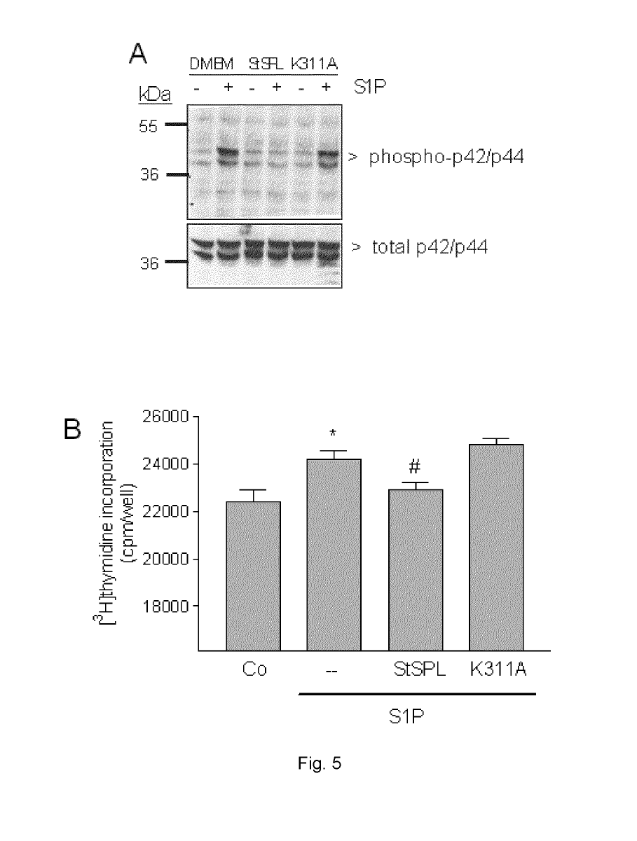

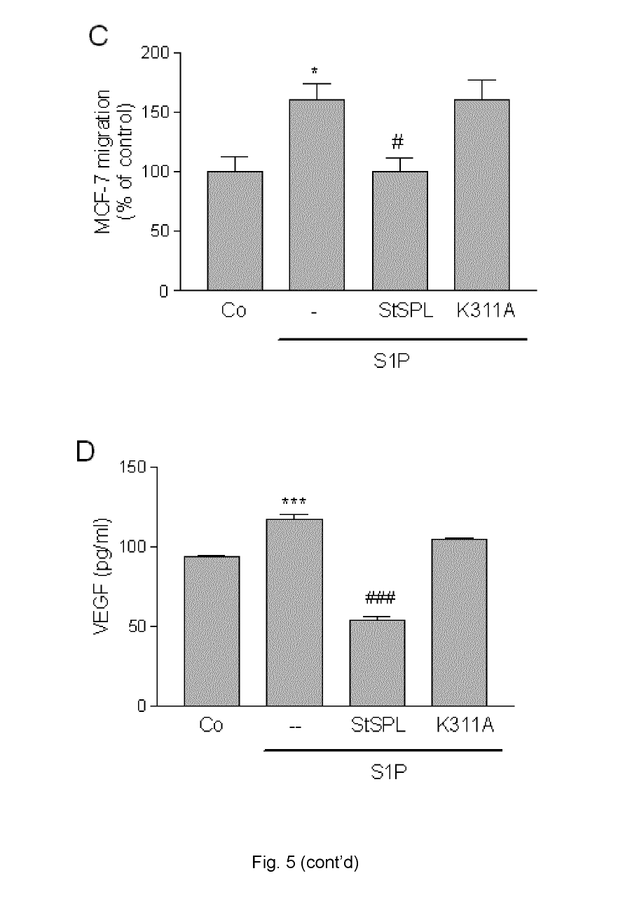

FIG. 5: Effect of StSPL on S1P-stimulated MAPK phosphorylation, proliferation, migration and VEGF production of MCF-7 breast carcinoma cells. (A) Quiescent MCF-7 cells were treated for 10 min with either vehicle (DMEM) or S1P (1 μM) in the absence or presence of wild-type StSPL (denoted StSPL, 10 μg/ml) or the K311A mutant (denoted K311A, 10 μg/ml). Cell lysates were prepared and separated by SDS-PAGE, transferred to nitrocellulose and subjected to Western blotting using antibodies against phospho-p42/p44 (dilution 1:1000, upper panel) and total p42/p44-MAPK (dilution 1:6000, lower panel). (B) Quiescent MCF-7 cells were treated for 24 h with either vehicle (Co) or S1P (1 μM), which had been pretreated for 30 min at 37° C. with either vehicle (−), wild-type StSPL (10 μg/ml) or a K311A mutant (10 μg/ml), in the presence of [³H]thymidine. Incorporated radioactivity was measured as described in the Methods Section. Results are expressed as cpm/well of incorporated [³H]thymidine and are means±S.D. (n=4). (C) Quiescent MCF-7 cells were treated for 24 h with DMEM (Co) or S1P (1 μM), which had been pretreated for 30 min at 37° C. with either vehicle (−), wild-type StSPL or the K311A mutant. Thereafter, migrated cells were analysed as described in the Methods Section. Results are expressed as migrated cells per counted field and are means±S.D. (n=3). (D) Quiescent MCF-7 cells were treated for 24 h with DMEM (Co) or S1P (1 μM) which had been pretreated for 30 min at 37° C. with either vehicle (−), wild-type StSPL (10 μg/ml), or the K311A mutant (10 μg/ml). Thereafter, supernatants were taken for a VEGF ELISA. Results are expressed as pg/ml of VEGF and are means±S.D. (n=4). *p<0.05, ***p<0.001 considered statistically significant when compared to the vehicle treated control values; #p<0.05, ###p<0.001 when compared to the S1P-treated values.

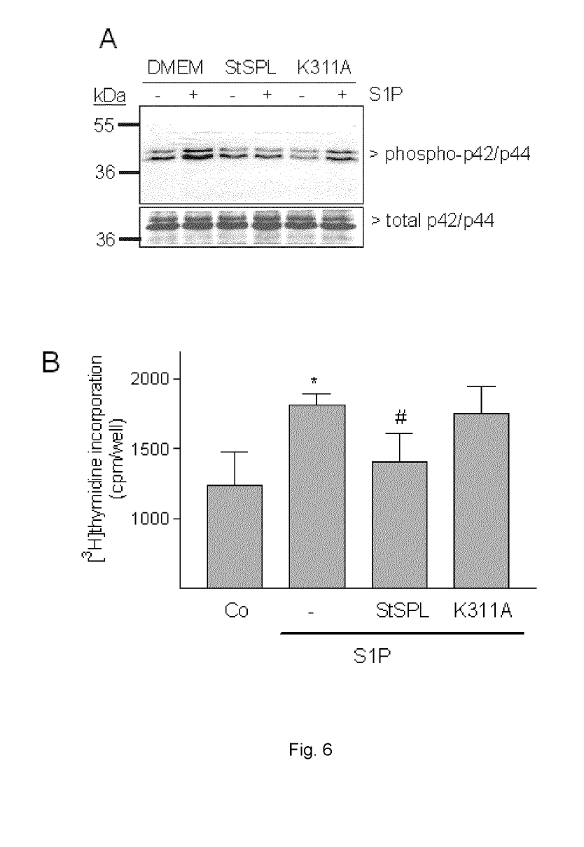

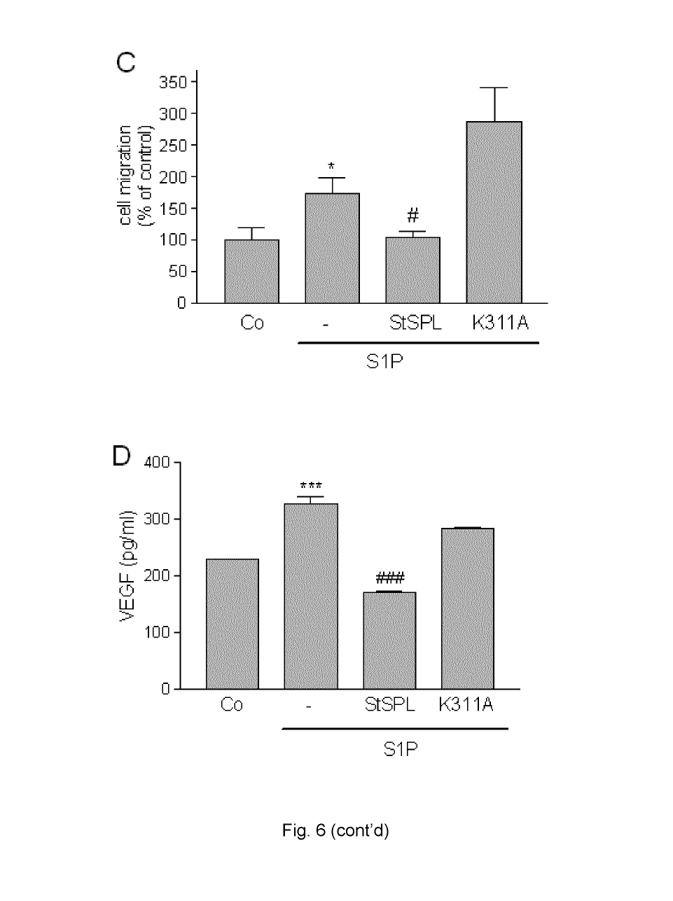

FIG. 6: Effect of StSPL on S1P-stimulated MAPK phosphorylation, proliferation and migration and VEGF synthesis in HCT-116 colon carcinoma cells. (A) Quiescent HCT-116 cells were treated for 10 min with either vehicle (DMEM, −) or S1P (1 μM) in the absence or presence of wild-type StSPL (denoted StSPL, 10 μg/ml) or the K311A mutant (denoted K311A, 10 μg/ml). Cell lysates were prepared and separated by SDS-PAGE, transferred to nitrocellulose and subjected to Western blotting using antibodies against phospho-p42/p44 (dilution of 1:1000, upper panel) and total p42/p44-MAPK (dilution each 1:6000, lower panel). (B) Quiescent HCT-116 cells were treated for 28 h with either vehicle (Co) or S1P (1 μM), which had been pretreated for 30 min at 37° C. with either vehicle (−), wild-type StSPL (10 μg/ml) or a K311A mutant (10 μg/ml), in the presence of [³H]thymidine. Incorporated radioactivity was measured as described in the Methods Section. Results are expressed as cpm/well of incorporated [³H]thymidine and are means±S.D. (n=4). (C) Quiescent HCT-116 cells were treated for 14 h with DMEM (Co) or S1P (1 μM), which had been pretreated for 30 min at 37° C. with either vehicle (−), wild-type StSPL or the K311A mutant. Thereafter, migrated cells were analysed as described in the Methods Section. Results are expressed as migrated cells per counted field and are means±S.D. (n=3). (D) Quiescent HCT-116 cells were treated for 14 h with DMEM (Co) or S1P (1 μM) which had been pretreated for 30 min at 37° C. with either vehicle (−), wild-type StSPL (10 μg/ml), or a K311A mutant (10 μg/ml). Thereafter, supernatants were taken for a VEGF ELISA. Results are expressed as pg/ml of VEGF and are means±S.D. (n=4). *p<0.05, ***p<0.001 considered statistically significant when compared to the vehicle treated control values; #p<0.05, ###p<0.001 when compared to the S1P-treated values.

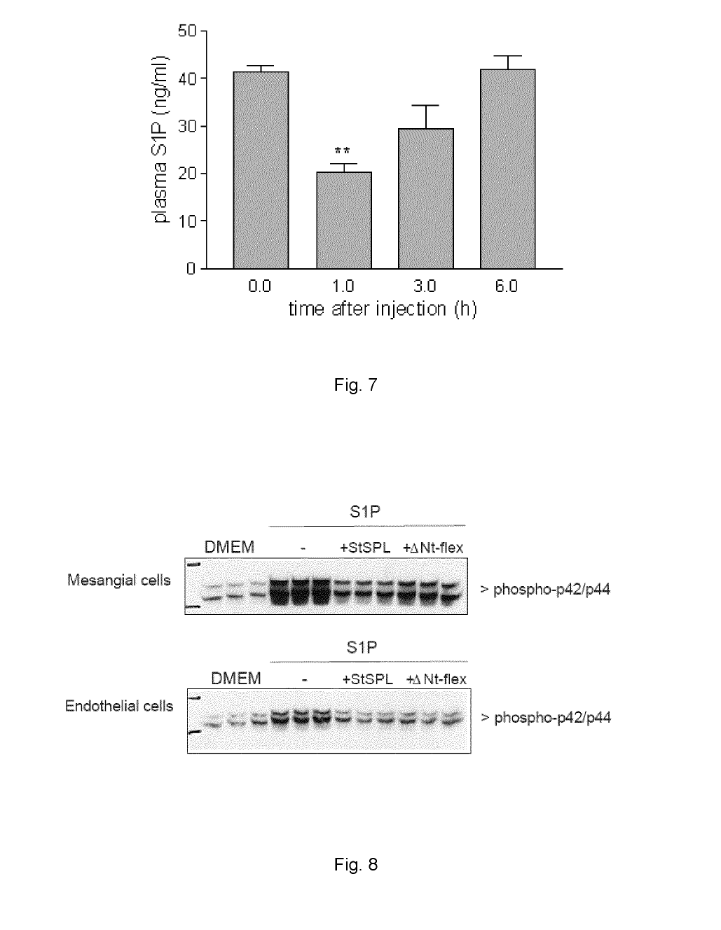

FIG. 7: In vivo activity of intravenously injected wild-type StSPL in mice. Wild-type S1P lyase (200 μg in 100 μl PBS per mouse) was injected intravenously into nude mice (n=4). Blood aliquots were taken from a lateral tail vein either before injection (0) or after 1 h, 3 h and 6 h. Plasma was prepared as described. 15 μl of plasma was subjected to lipid extraction as described in the Methods Section. S1P was quantified by LC/MS/MS as described. Data are expressed as ng/ml S1P and are means S.D. (n=4). **p<0.01 considered statistically significant when compared to the control values.

FIG. 8: Effect of wild-type StSPL versus the SPL variant ΔNt-FLEX lacking residues 1 to 57 on S1P-stimulated p42/p44-MAPK phosphorylation. Quiescent rat mesangial cells (upper panel) and human endothelial cells (lower panel) were treated for 10 min with either vehicle (DMEM) or S1P (1 μM) in the absence (−) or presence of wild-type StSPL (StSPL; 20 μg/ml) or the ΔNt-FLEX variant (20 μg/ml). Thereafter, cell lysates were separated by SDS-PAGE, transferred to nitrocellulose and subjected to Western blotting using antibodies against phospho-p42/p44 (dilution 1:1000). Blots were stained by the ECL method according to the manufacturer's recommendation. Data are representative of two independent experiments performed in triplicates.

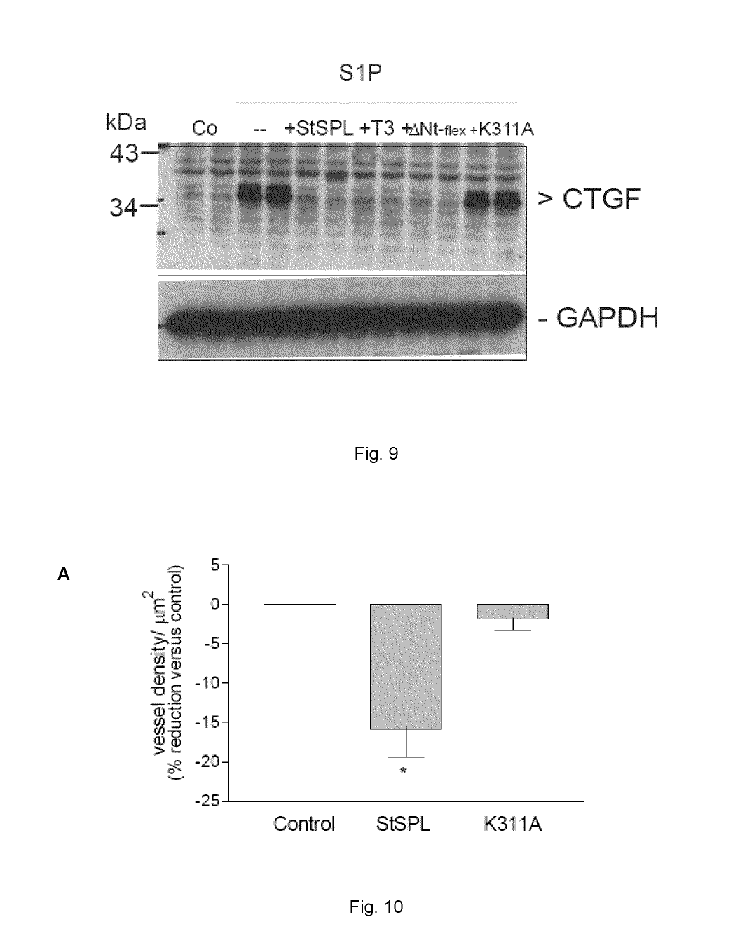

FIG. 9: Effect of wild-type StSPL versus SPL variant ΔNt-FLEX lacking residues 1 to 57 on S1P-stimulated CTGF expression in mouse fibroblasts. Quiescent mouse embryonic fibroblasts were treated for 4 h with either vehicle (Co) or S1P (1 μM) in the absence (−−) or presence of wild-type StSPL (StSPL; 20 μg/ml) or the ΔNt-FLEX variant (20 μg/ml). The mutant T3, in which 3 Tyr residues were mutated, as well as the mutant K311A lacking the pyridoxal-5′-phosphate binding site are shown as further controls. Thereafter, cell lysates were separated by SDS-PAGE, transferred to nitrocellulose and subjected to Western blotting using a CTGF-specific antibody (dilution 1:1000). Blots were stained by the ECL method according to the manufacturer's recommendation. Data are representative of two independent experiments performed in triplicates.

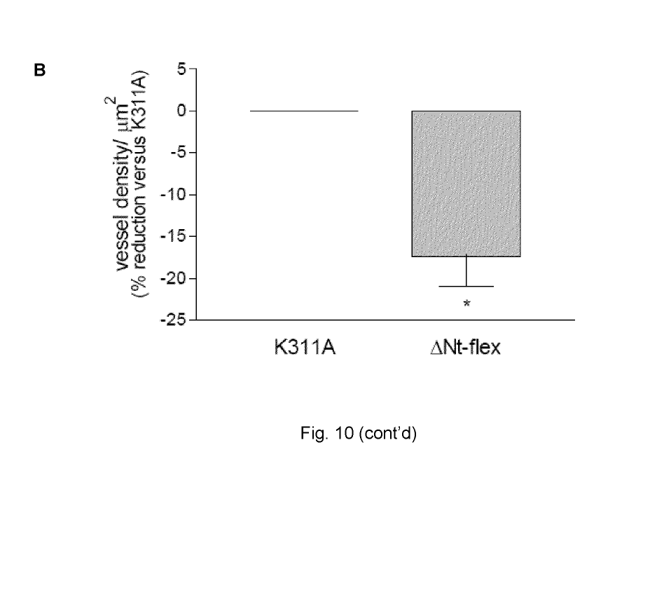

FIG. 10: In vivo effect of wild-type StSPL and the SPL variant ΔNt-FLEX lacking residues 1 to 57 on angiogenesis in the chicken chorioallantoic membrane (CAM) model. MCF-7 cell spheroids containing 5×10⁵ cells in 50 μl were placed on E8 CAMs, and either treated with PBS (control) (A), wild-type StSPL (StSPL, 20 μg/ml) (A), K311A mutant (20 μg/ml) (A,B), or the ΔNt-FLEX variant (20 μg/ml) (B) for 4 days. CAMs were analysed for vessel formation and the density of vessels per μm² of area around the tumor was determined using the free Vessel_tracer software. *p<0.05 was considered statistically significant when compared to the control treated samples (in A) and compared to the K311A-treated samples (in B).

The present invention is further illustrated by the following non-limiting examples.

### EXAMPLES

### Example 1

### Methods

Chemicals and Materials

Secondary horseradish peroxidase-coupled IgGs, Hyperfilm MP and enhanced chemiluminescence reagents were from GE Health-care Systems (Glattbrugg, Switzerland). S1P, C17-S1P, C17-sphingosine and C17-ceramide were from Avanti Polar Lipids (Alabaster, Ala., US). The antibody against phospho-p42/p44-mitogen-activated protein kinase (MAPK) was from Cell Signaling (Frankfurt am Main, Germany), antibodies against GAPDH (V-18) and connective tissue growth factor (CTGF) (L-20) were from Santa Cruz Biotechnology (Heidelberg, Germany), the total p42- and p44-MAPK antibodies were generated as previously described (Huwiler et al., 1994). The vascular endothelial growth factor (VEGF) enzyme-linked immunosorbent assay (ELISA) was from R&D Systems Europe Ltd. (Abingdon, U.K.). All cell culture additives were from Invitrogen AG (Basel, Switzerland).

Expression of Recombinant Wild-Type StSPL, the ΔNt-FLEX Variant Lacking Residues 1 to 57 and the K311A Mutant in E. coli

The recombinant wild-type StSPL and the K311A mutant lacking the pyridoxal-5′-phosphate binding site were expressed in E. coli and purified as described previously (Bourquin et al. (2010), supra). The in vitro activity of StSPL was monitored using a spectrophotometric and a mass spectrometric activity assay as two complementary activity assays. The first one undirectly monitors the cleavage of S1P while the second one directly records the cleavage of S1P (see Bourquin et al. (2010), supra).

Cell Culture

Rat renal mesangial cells were isolated and characterized as previously described (Pfeilschifter et al. (1984) Biochem J. 223:855-859). The human endothelial cell line EA.hy 926 was obtained from Dr. Edgell (Chapel Hill, N.C., USA) and was cultured as previously described (Schwalm et al. (2008) Biochem Biophys Res Commun 368(4): 1020-1025). MCF-7 breast carcinoma cells were cultured in Dulbecco's modified Eagle medium (DMEM) including 10% (v/v) fetal bovine serum, 6 μg/ml insulin, 100 units/ml penicillin, and 100 μg/ml streptomycin. HCT-116 colon carcinoma cells were cultured in McCoy medium including 10% (v/v) fetal bovine serum, 100 units/ml penicillin, and 100 μg/ml streptomycin. Prior to S1P stimulation, cells were rendered quiescent for 24 h in DMEM (for carcinoma cells phenolred-free medium was used) including 0.1 mg/ml of fatty acid-free bovine serum albumin (BSA).

Western Blotting

Stimulated cells were homogenised in lysis buffer and centrifuged for 10 min at 14000×g. The supernatant was taken for protein determination. 30 μg of protein were separated by SDS-PAGE, transferred to nitrocellulose membrane and subjected to Western blotting as previously described (Doll et al. (2005) Biochim Biophys Acta 1738(1-3): 72-81) using antibodies as indicated in the figure legends. For the detection of secreted CTGF, equal volumes of supernatants of stimulated cells were taken and proteins were precipitated with 7% trichloroacetic acid.

Quantification of S1P by Liquid Chromatography/Tandem Mass Spectrometry (LC/MS/MS)

15 μl of plasma samples or 100 μl of medium were taken for lipid extraction according to Bligh and Dyer ((1995) Can J Biochem Physiol 37(8): 911-917), and lipids were quantified by LC/MS/MS as described in Schmidt et al. (2006) Prostaglandins Other Lipid Mediat 81(3-4): 162-170.

[³H] Thymidine Incorporation into DNA

Confluent cells were starved for 24 h in serum-free DMEM containing 0.1 mg/ml of BSA. Thereafter, cells were stimulated in the presence of [³H]methyl-thymidine (1 μCi/ml) in the absence or presence of S1P, which had been preincubated for 30 min with either vehicle, wild-type StSPL or the K311A mutant, and StSPL was added for further 24-28 h. Cells were processed as described in Doll et al. (2005), supra.

Migration Assay

To measure undirected cell migration, an adapted Boyden chamber assay was performed as described in Schwalm et al. (2008), supra.

Quantification of VEGF

Secretion of VEGF into cell culture medium was quantified by ELISA (R&D Systems Europe Ltd., Abingdon, U.K.) according to the manufacturer's instructions. Confluent cells in 24-well-plates were stimulated in a volume of 0.5 ml.

In Vivo Activity of StSPL

Experiments were approved by the commission for animal experimentations of the Veterinäramt of the Kanton Berne. 10 week old female CD1 mice (Charles River, Sulzfeld, Germany) were injected intravenously with 200 μg wild-type StSPL in 100 μl PBS. Blood was taken either before treatment (control) or 1 h, 3 h and 6 h after injection by collecting 100 μl blood from the lateral tail vein using a heparinised capillary. Samples were centrifuged for 10 min at 2000×g and the supernatant (plasma) was taken for further quantification of S1P by LC/MS/MS.

Statistical Analysis

Statistical analysis was performed by one-tailed or two-tailed t-test. For further details, see also the above description of the figures.

### Example 2

### Biochemical Characterization of Recombinant StSPL

S1P lyase is the endogenous enzyme responsible for the irreversible degradation of S1P. In mammalian cells, the enzyme is normally located intracellularly at the ER membrane with its active site facing the cytosol. The main function of SPL is therefore to degrade intracellular S1P.

The product of the gene STH1274 from the thermophilic bacterium Symbiobacterium thermophilum, identified by bioinformatics analysis as a sphingosine-1-phosphate lyase, is an ortholog of Saccharomyces cerevisiae dihydrosphingosine-1-phosphate lyase (Dpl1p) (Bourquin et al. (2010, supra). The product of the gene STH1274 was named StSPL. The full-length STH1274 gene was cloned and expressed in E. coli and StSPL was purified to homogeneity as described in Bourquin et al. (2010), supra. A StSPL monomer is a 507 amino acid protein with a calculated molecular weight of 55 kDa which was detected at the expected size in a Coomassie stained SDS-PAGE (FIG. 1A, lane 2) and by Western blotting following protein migration on SDS-PAGE (FIG. 1A, lane 3). The structure of StSPL was solved using X-Ray diffraction. Full-length wild-type StSPL is a typical type I-fold dimeric pyridoxal-5′-phosphate (PLP)-dependent enzyme (FIG. 1B) in which residues from both subunits contribute to each active site of one subunit. A phosphate ion coming from the buffer (dot in FIG. 1B) sits near the cofactor PLP (hexagon in FIG. 1B) in the active site, mimicking the binding of the phosphate head of the substrate S1P. The stretch spanning residues 1 to 57 (named Nt-FLEX) was not visible in the electron density map due to disorder. Wild-type StSPL was shown to be active in vitro using two complementary activity assays. The first spectrophotometric assay indirectly monitored the cleavage of the S1P substrate by recording spectrophotometric changes of the cofactor upon catalysis (Bourquin et al., 2010). After addition of S1P to wild-type StSPL, the initial broad peak at 420-460 nm transiently disappeared and was replaced by a double peak at 420 & 403 nm (FIG. 1C). The visible spectrum of the inactive K311A mutant or of an inhibited wild-type StSPL did not undergo any changes upon addition of substrate. The second mass spectrometric activity assay monitors the disappearance of the S1P peak at m/z=380.26 after incubation with wild-type StSPL (FIG. 1D).

### Example 3

### StSPL is Active Under Extracellular Conditions

To investigate, whether StSPL is active also in the extracellular environment in the absence of pyridoxal-5′-phosphate, the enzyme was added to a cell culture medium supplemented with S1P and incubated at 37° C. As shown in FIG. 2A, S1P was degraded by 70% within 30 min, suggesting that even under extracellular conditions S1P is enzymatically degraded. In contrast, the K311A mutant of StSPL, which lacks the catalytically essential Schiff base bond with pyridoxal-5′-phosphate did not reduce the S1P levels in the culture medium (FIG. 2A).

To see whether StSPL is also active in blood and capable of degrading blood-derived S1P, human plasma was prepared from healthy donors and incubated in vitro with wild-type StSPL or the K311A mutant. As shown in FIG. 2B, incubation of plasma with buffer only at 37° C. did not alter the S1P level over a time period of 24 h. Moreover, there was no increase of sphingosine over 24 h of incubation (data not shown). These data demonstrate that S1P is rather stable in plasma depleted of blood cells, and exclude the spontaneous hydrolysis of S1P or an active degradation by other plasma factors such as plasma phosphatases. Incubation of plasma samples with wild-type StSPL rapidly degraded blood-derived S1P within 1 h of incubation, whereas control incubation with K311A did not affect S1P levels (FIG. 2B).

### Example 4

### StSPL Disrupts S1P-Stimulated Proliferation and Fibrotic Response in Renal Mesangial Cells

To analyse the biological effects of StSPL on renal mesangial cells as an in vitro model mimicking glomerular fibrosis, we tested the activity of purified StSPL on intact cells and assessed its ability to interfere with S1P signalling. To this end, we first tested renal mesangial cells, since in these cells S1P-triggered responses are well defined. The stimulation of mesangial cells with S1P for 10 min resulted in an increased phosphorylation and thus activation of the classical p42- and p44-MAPK/ERKs (FIG. 3A, upper panel). In the presence of wild-type StSPL, the S1P-triggered phosphorylation of p42- and p44-MAPKs was prevented, whereas the K311A mutant had no effect on the S1P-stimulated MAPKs (FIG. 3A).

S1P acts as a mitogen in renal mesangial cells (Hanafusa et al. (2002) Nephrol Dial Transplant 17(4): 580-586; Katsuma et al. (2002). Genes Cells 7(12): 1217-1230). and induces fibrosis as shown by upregulation of connective tissue growth factor (CTGF) (Xin et al. (2006) Br J Pharmacol 147(2): 164-174; Xin et al. (2004) J Biol Chem 279(34): 35255-35262), which represents a marker of fibrotic responses in vivo (Gellings Lowe et al. (2009) Cardiovasc Res 82(2): 303-312; Phanish et al. (2005) Nephron Exp Nephrol 100(4): e156-165). Mesangial cell proliferation was measured by [³H]thymidine incorporation into de-novo synthesized DNA. Treatment of quiescent mesangial cells with S1P for 28 h induced a moderate but significant increase in cell proliferation (FIG. 3B), which was prevented by wild-type StSPL but not the K311A mutant (FIG. 3B). It was previously demonstrated that S1P activates gene transcription and de-novo protein synthesis of pro-fibrotic CTGF in mesangial cells (Xin et al. (2004), supra). As shown in FIG. 3C, this effect of S1P was also prevented by wild-type StSPL, but not K311A.

These data suggest that extracellular StSPL not only abolishes S1P-mediated effects on acute cellular signalling cascades, but also reduces S1P-triggered cell responses such as proliferation and fibrotic reactions in cell culture models.

### Example 5

### StSPL Disrupts S1P-Stimulated Proliferation and Migration of Endothelial Cells

As an in vitro model of diseases associated with aberrant angiogenesis, the effect of StSPL on the human endothelial cell line EA.hy 926 was investigated. Again, S1P stimulated classical p42/p44-MAPKs phosphorylation, which was blocked by wild-type StSPL but not the K311A mutant (FIG. 4A).

In endothelial cells, S1P stimulates molecular events underlying angiogenesis which includes cell proliferation and migration (Folkmann et al. (2007) Nat Rev Drug Discov 6(4): 273-286). According to the present invention, it was found that S1P stimulated EA.hy 926 cell proliferation (FIG. 4B), which was impeded by wild-type StSPL but not K311A (FIG. 4B). Moreover, undirected endothelial cell migration was also stimulated by S1P as measured in an adapted Boyden chamber assay (FIG. 4C), and this effect was similarly prevented by wild-type StSPL but not K311A (FIG. 4C). In addition to migration, wild-type StSPL also reduced S1P-stimulated VEGF secretion in EA.hy 926 cells (FIG. 4D). These data strongly suggest that StSPL has potential to combat aberrant angiogenesis commonly associated with diseases like cancer, diabetic retinopathy and macular degeneration.

### Example 6

### StSPL Disrupts S1P-Stimulated Malignant Responses in Breast and Colon Carcinoma Cells

There is ample evidence that S1P contributes to tumorigenesis and malignant progression by promoting cell growth and metastasis (Pyne et al. (2010), supra). Therefore, we investigated whether StSPL can also attenuate S1P-stimulated cell responses in tumor cells like the breast carcinoma cell line MCF-7 and the colon carcinoma cell line HCT-116. As shown in FIGS. 5A and 6A, in both cell lines S1P stimulated classical p42/p44-MAPKs phosphorylation, which was prevented by wild-type StSPL but not the K311A mutant. Moreover, both cell lines responded to S1P stimulated by [³H]thymidine incorporation into DNA and this effect was again specifically impeded by StSPL (FIGS. 5B and 6B). Similarly, S1P stimulated migration of MCF-7 (FIG. 5C) and HCT-116 (FIG. 6C) cells, and this effect was also impeded by StSPL. In addition to migration, wild-type StSPL drastically reduced S1P-stimulated VEGF secretion in MCF-7 (FIG. 5D) and HCT-116 (FIG. 6D) cells.

These findings demonstrate the ability of StSPL to effectively impede also the pro-malignant effect of S1P on carcinoma cells.

### Example 7

### StSPL is Active In Vivo and Decreases Plasma S1P Levels in Mice

To investigate whether StSPL is also active under extracellular conditions in vivo, the enzyme was injected in mice and the degradation of S1P in mouse plasma was measured. As shown in FIG. 7, 1 h after injection of wild-type StSPL plasma S1P levels (determined as 40 ng/ml) decreased to about 70%. After 3 h, S1P levels were partly recovered and normal control levels were reached 6 h after injection (FIG. 7). This clearly demonstrates that recombinant wild-type StSPL retains its enzymatic acitivity also in vivo upon intravenous injection. On the other hand, it indicates that the S1P blood pool was effectively replenished by continuous production in blood cells and that StSPL was eliminated from the circulation.

### Example 8

### The ΔNt-FLEX variant of StSPL Lacking Residues 1 to 57 Reduces Early Signalling to the Same Extent as the Wild-Type StSPL

The full-length StSPL contains at its N-terminus a flexible sequence of 57 amino acids instead of the transmembrane sequence found in human, mouse and yeast SPL. In order to show that this N-terminal sequence is not required for StSPL activity and that thus a variant of StSPL lacking residues 1 to 57 (ΔNt-FLEX) has similar enzymatic activity as the wild-type, the effect of both wild-type StSPL and the StSPL ΔNt-FLEX variant on S1P-stimulated p42/p44-MAPK phosphorylation was investigated. To this purpose, quiescent rat mesangial cells (see FIG. 8, upper panel) and human endothelial cells (see FIG. 8, lower panel) were treated for 10 minutes with either vehicle (DMEM) or S1P (1 μM) in the absence (−) or presence of wild-type StSPL (StSPL; 20 μg/ml) or the ΔNt-FLEX variant (20 μg/ml). In the next step cells were lysed and the lysates were separated by SDS-PAGE, transferred to nitrocellulose and subjected to Western blotting using antibodies against phospho-p42/p44 (dilution 1:1000). Afterwards, the blots were stained by the ECL method according to the manufacturer's recommendation. As shown in FIG. 8 the ΔNt-FLEX variant shows a similar in vitro activity as the full-length StSPL and reduces early signalling such as S1P-stimulated p42/p44-MAPK phosphorylation and activation in renal mesangial cells and human endothelial cells (EA.hy 926).

### Example 9

### The ΔNt-FLEX Variant of StSPL Lacking Residues 1 to 57 Reduces S1P-Stimulated CTGF Expression to the Same Extent as the Wild-Type StSPL

To investigate whether the deletion of the first 57 amino acids of StSPL has any effect on the S1P-stimulated CTGF expression and secretion in mouse fibroblasts, quiescent mouse embryonic fibroblasts were treated for 4 h with either vehicle (Co) or S1P (1 μM) in the absence (−−) or presence of wild-type StSPL (StSPL; 20 μg/ml) or the ΔNt-FLEX variant (20 μg/ml) (see FIG. 9). The mutant T3, in which 3 Tyr residues were mutated into Phe as well as the mutant K311A lacking the pyridoxal-5′-phosphate binding site were used as controls. Cell lysates were separated by SDS-PAGE, transferred to nitrocellulose and subjected to Western blotting using a CTGF-specific antibody (dilution 1:1000). In the next step, blots were stained by the ECL method according to the manufacturer's recommendation. FIG. 9 shows that CTGF-levels in the cell lysates of cells that have been treated with S1P in the presence of wild-type StSPL or the ΔNt-FLEX variant, respectively, are comparable. Therefore, in mouse fibroblasts, S1P-stimulated CTGF expression and secretion is reduced by the ΔNt-FLEX variant in a similar manner as by the wild-type StSPL (see FIG. 9), suggesting that the ΔNt-FLEX variant has a comparable anti-fibrotic potential as the wild-type.

### Example 10

### In Vivo Effect of the StSPL Variant ΔNt-FLEX Lacking Residues 1 to 57: Reduction of Neovascularization

The in vivo effect of wild-type StSPL and of the ΔNt-FLEX variant on angiogenesis was investigated using tumors cells growing on the chorioallantoic membrane (CAM) of developing chicken embryos. Fertilized chicken eggs (Bruterei E. Wüthrich A G, Belp, Switzerland) at embryonic day 4 (E4) were opened and placed into plastic dishes (Thermoflex A G, Switzerland) and further incubated at 37° C. and 55% relative humidity. At E8, 5×10⁵ MCF-7 cell spheroids, which were prepared in growth medium containing 0.2% methylcellulose, were placed on the CAM and either treated with PBS (control) (see FIG. 10A), wild-type StSPL (StSPL, 20 μg/ml) (see FIG. 10A), K311A mutant (20 μg/ml) (see FIGS. 10A and B) or the ΔNt-FLEX variant (20 μg/ml) (see FIG. 10B) for 4 days. At E12, CAMs were examined for vessel formation under a stereomicroscope (Carl Zeiss A G, Feldbach, Switzerland). The density of vessels per area around the tumor was determined using the free downloadable software Vessel_tracer developed by Sofka and Stewart (Sofka and Stewart (2006) IEEE transactions on medical imaging 25: 1531-1546) (http://www.cs.rpi.edu/˜sofka/vessels_exec.html). *p<0.05 was considered statistically significant when compared to the control-treated samples (in FIG. 10A) and compared to the K311A-treated samples (in FIG. 10B). It is shown that treatment of MCF-7 spheroids with wild-type StSPL for 4 days reduced vessel formation by approx. 18% compared to buffer-treated CAMs (see FIG. 10A), and the same effect of 18% reduction of neovascularization is demonstrated for the ΔNt-FLEX variant. The inactive K311A mutant was ineffective.

1. A transmembrane domain-free sphingosine-1-phosphate lyase (S1PL) or functional derivative or mutant thereof or a nucleic acid encoding a transmembrane domain-free S1PL or a functional derivative or mutant thereof for the prevention or treatment of a pathologic condition associated with elevated levels of sphingosine-1-phosphate (S1P).

2. The transmembrane domain-free S1PL or nucleic acid of claim 1 wherein the S1PL or a nucleic acid coding therefore is derived from a bacterium or an amoeba.

3. The transmembrane domain-free S1PL or nucleic acid of claim 2 wherein the bacterium is selected from the group consisting of Symbiobacterium thermophilum, Erythrobacter litoralis, Myxococcus xanthus, Burkholderia thailandensis, Burkholderia pseudomallei, Erythrobacter sp., Myxococcus fulvus, Streptomyces sp., Stigmatella aurantiaca, Rhodococcus erythropolis, Plesiocystis pacifica and Fluoribacter dumoffii.

4. The transmembrane domain-free S1PL or nucleic acid of claim 1 wherein the S1PL is selected from the group consisting of SEQ ID NO: 1 to 26, 28 and 36 or wherein the nucleic acid comprises a nucleotide sequence encoding an amino acid sequence selected from the group consisting of SEQ ID NO: 1 to 26, 28 and 36.

5. The transmembrane domain-free S1PL or nucleic acid of claim 2 wherein the amoeba is Polysphondylium pallidum.

6. The transmembrane domain-free S1PL or nucleic acid of claim 5 wherein the S1PL has an amino acid sequence of SEQ ID NO: 27 or wherein the nucleic acid comprisies a nucleotide sequence coding for an amino acid sequence of SEQ ID NO: 27.

7. The transmembrane domain-free S1PL or nucleic acid according to claim 1 wherein the pathologic condition is selected from the group consisting of hyperproliferative diseases, inflammation, autoimmune diseases, diabetic retinopathy and macular degeneration.

8. The transmembrane domain-free S1PL or nucleic acid of claim 7 wherein the hyperproliferative disease is selected from the group consisting of cancer, fibrosis and aberrant angiogenesis.

9. A transmembrane domain-free sphingosine-1-phosphate lyase (S1PL) or functional derivative or mutant thereof or a nucleic acid encoding a transmembrane domain-free S1PL or a functional derivative or mutant thereof for use as a medicament.

10. The transmembrane domain-free S1PL or nucleic acid of claim 9 wherein the S1PL or nucleic acid is selected from the group consisting of SEQ ID NO: 1 to 28, and 36.

11. A method for the prevention or treatment of a pathologic condition associated with elevated levels of sphingosine-1-phosphate (S1P) comprising the step of administering to a patient in need thereof a therapeutically effective amount of a transmembrane domain-free sphingosine-1-phosphate lyase (S1PL) or functional derivative or mutant thereof or a nucleic acid encoding a transmembrane domain-free S 1PL or a functional derivative or mutant thereof.

12. The method of claim 11 wherein the S 1PL or nucleic acid is selected from the group consisting of SEQ ID NO: 1 to 28, and 36.

13. The method of claim 11 wherein the pathologic condition is selected from the group consisting of hyperproliferative diseases, inflammation, autoimmune diseases, diabetic retinopathy and macular degeneration.

14. The method of claim 13 wherein the hyperproliferative disease is selected from the group consisting of cancer, fibrosis and aberrant angiogenesis.

15. The method according to claim 11 further comprising administering a therapeutically effective amount of a transmembrane domain-free sphingosine-1-phosphate lyase (S1PL) intravenously.

16. A prokaryotic sphingosine-1-phosphate lyase (S1PL) containing a transmembrane domain or functional derivative or mutant thereof or a nucleic acid encoding a prokaryotic S1PL containing a transmembrane domain or a functional derivative or mutant thereof for the prevention or treatment of a pathologic condition associated with elevated levels of sphingosine-1-phosphate (S1P).

17. The S1PL containing a transmembrane domain of claim 16 wherein the S1PL containing a transmembrane domain is derived from a bacterium selected from the group consisting of Legionella pneumophila, Legionella jamestowniensis and the marine gamma proteobacterium HTCC2143.

18. The S1PL containing a transmembrane domain or nucleic acid of claim 16 wherein the S1PL is selected from the group consisting of SEQ ID NO:
31 to 35 or wherein the nucleic acid comprises a nucleotide sequence encoding an amino acid sequence selected from the group consisting of SEQ ID NO: 31 to 35.

19. The S1PL containing a transmembrane domain or nucleic acid according to claim 16 wherein the pathologic condition is selected from the group consisting of hyperproliferative diseases, inflammation, autoimmune diseases, diabetic retinopathy and macular degeneration.

20. The S1PL containing a transmembrane domain or nucleic acid of claim 19 wherein the hyperproliferative disease is selected from the group consisting of cancer, fibrosis and aberrant angiogenesis.

21. A prokaryotic sphingosine-1-phosphate lyase (S1PL) containing a transmembrane domain or functional derivative or mutant thereof or a nucleic acid encoding a prokaryotic S1PL containing a transmembrane domain or a functional derivative or mutant thereof for use as a medicament.

22. The prokaryotic S1PL or nucleic acid of claim 21 wherein the S1PL or nucleic acid is selected from the group consisting of SEQ ID NO: 31 to 35.

23. A method for the prevention or treatment of a pathologic condition associated with elevated levels of sphingosine-1-phosphate (S1P) comprising the step of administering to a patient in need thereof a therapeutically effective amount of a prokaryotic sphingosine-1-phosphate lyase (S1PL) containing a transmembrane domain or functional derivative or mutant thereof or a nucleic acid encoding a prokaryotic S1PL containing a transmembrane domain or a functional derivative or mutant thereof.

24. The method of claim 23 wherein the S 1PL is selected from the group consisting of SEQ ID NO: 31 to 35.

25. The method of claim 23 wherein the pathologic condition is selected from the group consisting of hyperproliferative diseases, inflammation, autoimmune diseases, diabetic retinopathy and macular degeneration.

26. The method of claim 25 wherein the hyperproliferative disease is selected from the group consisting of cancer, fibrosis and aberrant angiogenesis.

27. The method according to claim 23 further comprising administering a therapeutically effective amount of a prokaryotic sphingosine-1-phosphate lyase (S1PL) containing a transmembrane domain intravenously.

28. A transmembrane-free sphingosine-1-phosphate lyase (S1PL) lacking the N-terminal loop domain or a functional derivative or mutant thereof.

29. The S1PL lacking the N-terminal loop domain of claim 28 wherein the S1PL is derived from a bacterium selected from the group consisting of Symbiobacterium thermophilum, Erythrobacter litoralis, Myxococcus xanthus, Burkholderia thailandensis, Burkholderia pseudomallei, Erythrobacter sp., Myxococcus fulvus, Streptomyces sp., Stigmatella aurantiaca, Rhodococcus erythropolis, Plesiocystis pacifica and Fluoribacter dumoffii.

30. The S1PL lacking the N-terminal loop domain of claim 28 having an amino acid sequence selected from the group consisting of SEQ ID NO: 36 to 38.

31. A polynucleotide encoding the S1PL according to claim 28.

32. A vector containing the polynucleotide of claim 31.

33. A cell transformed with the polynucleotide of claim 31.

34. A method for the production of the S1PL according to claim 28 comprising the steps of:
(a) culturing cells transformed with a polynucleotide encoding an amino acid sequence selected from the group consisting of SEQ ID NO: 36 to 38 in a culture medium allowing the expression of said S1PL; and
(b) purifying said S1PL from the culture medium and/or the cells.

35. A pharmaceutical composition comprising the S1PL according claim 28 in combination with at least one pharmaceutically acceptable carrier, excipient and/or diluent.


SEQUENCE LISTING (38 sequences)

SEQ ID NO: 1 - protein, 507 aa, Symbiobacterium thermophilum
MLSAFSPPLPCDPARSHPTPEFPSSLQDYCEIRGIQSQPPARRDPTMDWLASLRSQIKPYRDRFPSHARLPRAGLPRAEILAEIAAMGAAESPAWRDGYASGAVYHGDEHHIAFLNEVYALQSQSNPLHPDLWPSTAKFEAEVVAMTAHMLGGDAAGGTVCGTVTSGGTESLLLAMKTYRDWARATKGITAPEAVVPVSAHAAFDKAAQYFGIKLVRTPLDADYRADVAAMREAITPNTVVVAGSAPGYPHGVVDPIPEIAALAAEHGIGCHVDACLGGFILPWAERLGYPVPPFDFRLEGVTSVSADTHKYGYGAKGTSVILYRRPDLLHYQYFIAADWPGGLYFSPTFAGSRPGALSATAWAAMLSLGEEGYLDATRRILQAADRLKAGVRAIPSLKILGDPLWVIAVASDELNIYQVMEEMAGRGWRLNGLHRPPAFHVALTLRHTEPGVVDRFLADLQDAVAQVRAHPEKATGMAPVYGMAAAAPPELVRQVLTGFIDLLYEV

SEQ ID NO: 2 - protein, 412 aa, Erythrobacter litoralis
MTMPKRGRDWAEVRAEMIDRGAGDAKWRDGKTAVYVFNAGPDIAEVQHEAYALYMSENGLGPLAFPSLAQMEREVIEMALSLLRGPEGAAGAMTSGGTDSITMAVKAARDFARAEKGLSGPANIVLPKSAHPAFDKAAHLMDIEVRRVPLKDDGSYEADPAAMDAACDAATIMMVGSAPNFPHGIVDPIMTLGEVAQARDIWLHTDACVGGYFAPFARMNGVDVPPFDFEVPAVRSMSADLHKYGYAAKGASTVLFRSEEYYNHMPFENRDWSGAPMKTPTLAGTRPGGAISAAWAVMQVLGVEGYREKQGLVCATRERVEAGVRELGFEVLGNPLLGLIAFRHPEHDNYAIYSEMYRKGWFTSLTIEPPALHLMLSPKHAEVIDDYLVDLAAGLETVAAGKEGPQVEARYN

SEQ ID NO: 3 - protein, 509 aa, Myxococcus xanthus
MALPDLKALSLLNHVPPRLLSAAERYLKAVPGVKGLLNRETGSLLSELESGLKPYRGKMQSFDHLPPTGRSREDVLRELQALESQEEYRWREGRVSGGVYNGDAEHIAFLNRVYALHSQSNPLHADLWPSATKFEAEVVAMTASMLGADVANAGQPEEARICGAMSSGGTESIMLAMKTYRDWARETKGITRPEMVAPASAHPAFDKAAHYFGIKMVRVPVGPDYRADVAAMRKAINRNTIVLIGSAPGFPHGVIDPIEALSEIARKKRIGFHTDACLGGFVLPFAMKLGYDVPPFDFRLPGVTSMSVDTHKFGYAAKGSSVVLYRGTELRSHQYFTATDWPGGIYFSPTFSGSRPGALIASAWAALVSTGEQGYLDATRSILETAAAIKKGIRDIPELHVLGDPLFVIAFGSESVDIFQVMERMSEHGWNLNGLHKPSAVHLCVTLRHTQPGVAEQFLVDLRDAVDYVKSHPSAKGTMAPVYGMAASVPFRGLVSDLLKKYMDLLYKV

SEQ ID NO: 4 - protein, 473 aa, Burkholderia thailandensis
MDLEEGIRRLYPYAEEFGVTRGFPEHGMPRDALLAQLRSMAEREDRNWESGRCSGAMYSGDRDHHAWLNEAYSIFSHVNALRRDMCPSMNRMESEIVGMTVAMLHGEAVAAHHPGQRACGMITLGGTESILGATLAYREKARAERGIDRPRMIWPASAHPVFRKAAHLFGFDVTVAPVDPDTMQVDADFVRDAIDADTVMLVGSACNYPYGTVDPIEALSEIAVEKEVWLHVDGCLGGWMLPWGEELGYPNIPAFDFRLPGVTSISADTHKFGYGPKGGSVLAWRDASFRRYQYFLMTDWVGGVYGSPGLAGSRSGGLIAATWAALRGLGREGYRARARAIFDTAFDMQAAVRAIPELRVLGKPTFCFAFASDAFDIYHVNDFMRQRGWRLNGLRHPDALQMCVTEPQTRPGVADQFRRDLGAAVEHARRHARERPKTSGIYASDATGVDLSDDARARAFFTQVIDLFTDCPL

SEQ ID NO: 5 - protein, 473 aa, Burkholderia thailandensis
MDLEEGIKRLYPYATEFGAMHGFPEHGLSRERLLDELRSMAVREDRKWESGRCSGTMYCGDHEHYAFLNEAYGLFSHVNALQRDLCPSMSRMESEIVGMTVAMLHGEAVAGHGGAHRACGVLSLGGTESILNATLAYREKARAERGIDRPRMIWPASAHPAFRKAAHLFGFDVTVAPVDPDTMQVDADFVRDAIDANTVMLVGSACNYPYGTVDPIEALSEIAVEKEVWLHVDGCLGGWMLPWGEELGYPNIPAFDFRLPGVTSISADTHKFGYGPKGGSVLAWRDASFRRYQYFLMTDWVGGVYGSPGLAGSRSGGLIAATWAALRGLGREGYRARARAIFDTAFDMQAAVRAIPELRVLGKPTFCFAFASDAFDIYHVNDFMRRQGWRFNGLQHPDALHMCVTGPQTQPGVAKRFRQDLGAAVDYARRHTHARAQSSGVYGGDSAGLDLRDDAQARAFFTQVLDLFTDCPL

SEQ ID NO: 6 - protein, 473 aa, Burkholderia pseudomallei
MDLEEGVRQLYPYAAEFGALHEFPERGMPRERLLEELRSMAVREDRKWESGRCSGTMYCGDHEHYAFLNEAYGLFSHVNALQRDLCPSMNRMESEIVAMTVALLHGEAVQRHDGAHRACGALSLGGTESILNATLAYREKARAERGIERPRMIWPASAHPAFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRAIPELRVLGKPTFCFAFTSDAFDIYHVNDFMRQRGWRFNGLQHPDALHMCVTGPQTQPGVAERFRQDLGEAVEHARHRAHARPQSSGVYGGDAAGLDLRDDARARAFFTQVLDLFTDCPL

SEQ ID NO: 7 - protein, 498 aa, Burkholderia pseudomallei
MQPHPGNIRTHIGVPTDVGTAKENVMDLEEGIRRLYPYAEEFGVMRGFPEQGTPRDELLAQLRSMAEREDRNWESGHSSGAMYSGDRDHHAWLNEAYSVFSHVNALRRDMCPSMNRMESEIVGMTVAMLHGDAVAAHHPGQRACGMITLGGTESILGATLAYREKARAERGIERPRMIWPASAHPVFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRAIPELRVLGKPTFCFAFTSDAFDIYHVNDFMRQRGWRLNGLRRPDALQMCVTGPQTQPGVAEQFRCDLGAAADYARAHAQERPKTSGVYASDAAGVDLSDDARTRAFFTQVIDLFTDCPL

SEQ ID NO: 8 - protein, 473 aa, Burkholderia pseudomallei
MDLEEGVRQLYPYAAEFGALHEFPERGMPRERLLEELRSMAVREDRKWESGRCSGTMYCGDHEHYAFLNEAYGLFSHVNALQRDLCPSMNRMESEIVAMTVALLHGEAVQRHDGAHRACGALSLGGTESILNATLAYREKARAERGIERPRMIWPASAHPVFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRAIPELRVLGKPTFCFAFTSDAFDIYHVNDFMRQRGWRFNGLQHPDALHMCVTGPQTQPGVAERFRQDLGEAVEHARHRAHARPQSSGVYGGDAAGLDLRDDARARAFFTQVLDLFTDCPL

SEQ ID NO: 9 - protein, 485 aa, Burkholderia pseudomallei
MPTDVGTAKENVMDLEEGIRRLYPYAEEFGVMRGFPEQGTPRDELLAQLRSMAEREDRNWESGHSSGAMYSGDRDHHAWLNEAYSVFSHVNALRRDMCPSMNRMESEIVGMTVAMLHGDAVAAHHPGQRACGMITLGGTESILGATLAYREKARAERGIERPRMIWPASAHPVFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRGLGREGYLARAKAIFETAFDMQAAVRAIPELRVLGKPTFCFAFTSDAFDIYHVNDFMRQRGWRLNGLRRPDALQMCVTGPQTQPGVAEQFRCDLGAAADYARAHAQERPKTSGVYASDAAGVDLSDDARTRAFFTQVIDLFTDCPL

SEQ ID NO: 10 - protein, 473 aa, Burkholderia pseudomallei
MDLEEGVRQLYPYAAEFGALHEFPERGMPRERLLEELRSMAVREDRKWESGRCSGTMYCGDHEHYAFLNEAYGLFSHVNALQRDLCPSMNRMESEIVAMTVALLHGEAVQRHDGAHRACGALSLGGTESILNATLAYREKARAERGIERPRMIWPASAHPAFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRAIPELRVLGKPTFCFAFTSDAFDIYHVNDFMRQRGWRLNGLRRPDALHMCVTGPQTQPGVAERFRQDLGEAVEHARHRAHARPQSSGVYGGDAAGLDLRDDARARAFFTQVLDLFTDCPL

SEQ ID NO: 11 - protein, 485 aa, Burkholderia pseudomallei
MPTDVGTAKENVMDLEEGIRRLYPYAEEFGVMRGFPEQGTPRDELLAQLRSMAEREDRNWESGHSSGAMYSGDRDHHAWLNEAYSVFSHVNALRRDMCPSMNRMESEIVGMTVAMLHGDAVAAHHPGQRACGMITLGGTESILGATLAYREKARAERGIERPRMIWPASAHPVFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRAIPELRVLGKPTFCFAFTSDAFDIYHVNDFMRQRGWRLNGLRRPDALQMCVTGPQTQPGVAEQFRCDLGAAADYARAHAQERPKTSGVYASDAAGVDLSDDARTRAFFTQVIDLFTDCPL

SEQ ID NO: 12 - protein, 473 aa, Burkholderia pseudomallei; identical to SEQ ID NO: 6

SEQ ID NO: 13 - protein, 485 aa, Burkholderia pseudomallei
MPTDVGTAKENVMDLEEGIRRLYPYAEEFGVMRGFPEQGTPRDELLAQLRSMAEREDRNWESGHSSGAMYSGDRDHHAWLNEAYSVFSHVNALRRDMCPSMNRMESEIVGMTVAMLHGDAVAAHHPGQRACGMITLGGTESILGATLAYREKARAERGIERPRMIWPASAHPVFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRAIPELRVLGKPTFCFAFASDAFDIYHVNDFMRQRGWRLNGLRRPDALQMCVTGPQTQPGVAEQFRCDLGAAADYARAHAQERPKTSGVYASDAAGVDLSDDARTRAFFTQVIDLFTDCPL

SEQ ID NO: 14 - protein, 485 aa, Burkholderia pseudomallei
MPTDVGTAKENVMDLEEGIRRLYPYAEEFGVMRGFPEQGTPRDELLAQLRSMAEREDRNWESGHSSGAMYSGDRDHHAWLNEAYSVFSHVNALRRDLCPSMNRMESEIVGMTVAMLHGDAVAAHHPGQRACGMITLGGTESILGATLAYREKARAERGIERPRMIWPASAHPVFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRGLGREGYLARAKAIFETAFDMQAAVRAIPELRVLGKPTFCFAFTSDAFDIYHVNDFMRQRGWRLNGLRRPDALQMCVTGPQTQPGVAEQFRCDLGAAADYARAHAQERPKTSGVYASDAAGVDLSDDARTRAFFTQVIDLFTDCPL

SEQ ID NO: 15 - protein, 507 aa, Burkholderia pseudomallei
MRVACFVDFMQPHPGNIRTHIGVPTDVGTAKENVMDLEEGIRRLYPYAEEFGVMRGFPEQGTPRDELLAQLRSMAEREDRNWESGHSSGAMYSGDRDHHAWLNEAYSVFSHVNALRRDMCPSMNRMESEIVGMTVAMLHGDAVAAHHPGQRACGMITLGGTESILGATLAYREKARAERGIERPRMIWPASAHPVFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRGLGREGYLARAKAIFETAFDMQAAVRAIPELRVLGKPTFCFAFASDAFDIYHVNDFMRQRGWRLNGLRRPDALQMCVTGPQTQPGVAEQFRRDLGAAADYARAHAQERPKTSGVYASDAAGVDLSDDARTRAFFTQVIDLFTDCPL

SEQ ID NO: 16 - protein, 493 aa, Burkholderia pseudomallei
MRSIAARSSHAGAGLEQEGVMDLEEGVRQLYPYAAEFGALHEFPERGMPRERLLEELRSMAVREDRKWESGRCSGTMYCGDHEHYAFLNEAYGLFSHVNALQRDLCPSMNRMESEIVAMTVALLHGEAVQRHDGAHRACGALSLGGTESILNATLAYREKARAERGIERPRMIWPASAHPAFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRGLGREGYLARAKAIFETAFDMQAAVRAIPELRVLGKPTFCFAFTSDAFDIYHVNDFMRQRGWRFNGLQHPDALHMCVTGPQTQPGVAERFRQDLGEAVEHARHRAHARPQSSGVYGGDAAGLDLRDDARARAFFTQVLDLFTDCPL

SEQ ID NO: 17 - protein, 493 aa, Burkholderia pseudomallei
MRSIAARSSHAGAGLEQEGVMDLEEGVRQLYPYAAEFGALHEFPERGMPRERLLEELRSMAVREDRKWESGRCSGTMYCGDHEHYAFLNEAYGLFSHVNALQRDLCPSMNRMESEIVAMTVALLHGEAVQRHDGAHRACGALSLGGTESILNATLAYREKARAERGIERPRMIWPASAHPAFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRAIPELRVLGKPTFCFAFASDAFDIYHVNDFMRQRGWRFNGLQHPDALHMCVTGPQTQPGVAERFRQDLGEAVEHARHRAHARPQSSGVYGGDAAGLDLRDDARARAFFTQVLDLFTDCPL

SEQ ID NO: 18 - protein, 493 aa, Burkholderia pseudomallei
MRSIAARSSHAGAGLEQEGVMDLEEGVRQLYPYAAEFGALHEFPERGMPRERLLEELRSMAVREDRKWESGRCSGTMYCGDHEHYAFLNEAYGLFSHVNALQRDLCPSMNRMESEIVAMTVALLHGEAVQRHDGAHRACGALSLGGTESILNATLAYREKARAERGIERPRMIWPASAHPAFRKAAHLFGFDVTVAPIDPVTMQVDADFVRDAVDANTVMLVGSACNYPYGTIDPIGALSAIAVEKDVWLHVDGCLGGWMLPWGEALGYPDIPAFDFRLPGVTSISADTHKFGYGPKGGSVLAWRDASFRRHQYFLMTDWVGGVYGSPGLTGSRSGGLIAATWAALRSLGREGYLARAKAIFETAFDMQAAVRSIPELRVLGKPTFCFAFASDAFDIYHVNDFMRQRGWRFNGLQHPDALHMCVTGPQTQPGVAEQFRQDLGEAVEHARHRAHARPQSSGVYGGDAAGLDLRDDARARAFFTQVLDLFTDCPL

SEQ ID NO: 19 - protein, 412 aa, Erythrobacter sp.
MAMPKAGRSWEDVREEMIARGAGDAKWRDGKTAVYVFNAGPEIGEVQHEAYTLFMSENGLGPLAFPSLAQMEREVVEMALSVLHGPEGATGAITSGGTDSITMAMKAARDYARAEKGLSGPANIVLPQSAHPAFDKAAHLMDIEVRRVPLKVDGSYEADPAAMGEACDQSTIMMVGSAPNFPHGIIDPIEALGQVAEAKGVWLHTDACVGGYFAPFARMNGVPVPPFDFEVTGVHSMSADLHKYGYAAKGASTVLFRSKALFEHMPFDTKNWNGAPMKTPTLAGTRPGGAISAAWAVMNVLGIEGYREKQGLVCETRERAEKGLAQLRFEVLGKPMLGLIAFRHPEHDSFALYSEMYRKGWFTSITIDPPSLHLMLSPKHAEVIDQYLDDLREACETVEAGKEGAKVEARYN

SEQ ID NO: 20 - protein, 509 aa, Myxococcus fulvus
MALPDLKALSLLNHVPPRLLSAAERYLKAVPGVKGLLNRETGTLLSELESGLKPYRGKMQSFDHLPPTGRSRDDVLRELQALESQEEARWREGRVSGGVYNGDAEHIAFLNRVYALHSQSNPLHADLWPSATKFEAEVVAMTASMLGADVANAGVPEEARICGAMSSGGTESIMLAMKTYRDWARENQGITKPEMVAPASAHPAFDKAAHYFGIKMVRVPVGPDYRADVAAMRKAVNRNTIVIIGSAPGFPHGVIDPIEALSELARKKRIGFHTDACLGGFVLPFAKKLGYDVPPFDFRLPGVTSMSVDTHKFGYAAKGSSVVLYRGTELRSHQYFTATDWPGGIYFSPTFSGSRPGALIASAWAALVCMGEQGYLDATRHILETAAAIKQGIRDIPELHVLGDPLFVIAFGSESLDIFQVMERMSEHGWNLNGLHKPSAVHLCVTLRHTQPGVAEQFLTDLRDAVDYVKSHPSAKGTMAPVYGMAASVPFRGLVSDLLKKYMDLLYKV

SEQ ID NO: 21 - protein, 503 aa, Streptomyces sp.
MLGGVWTPQEPDAAPSPSPDALPPGRPADELLAELRDLRTGDAPTRGGRTFAYVYDAGLPGLDDLSAAAYGTYATVNGLDPTVFPSVARLENDLVGAVTAVLGTPGAQGAFTSGGTESVLLAVKAARDHARTERGITEPQLVLPSTAHAAFHKAAHYLGLEAVTVPVDPVSFRAPAAAVEAALTERTALVVASAPSYAHGVMDPVTDIAAAAAARGILCHVDACIGGWFLPFLRRTGREVEPFDLSVPGVTSLSVDLHKYGYADKGASVILYRDAELRRHQYFAHAGWPGYPVVNPTVQGTKSAGLLAQAWAVLRHIGGDGYTALARRIADAADRLLPALRALPGVRVLGEPAGSLVAFTVTGADDAPDLARVLHLADEMRERGWHLQPQLSFGPLPPNLHLTLTPATNDRVEALLADLADAVKTVGASEPARPDPALAEFAAGLDPDRLGPEEVAAVLAFAGLDGSGPLPTRMAPVLMLLDTLPVRLKERLLTEFIGSLFRI

SEQ ID NO: 22 - protein, 440 aa, Stigmatella aurantiaca
MTSKSLPKNKRSKEEVLAELRTLRAEDARWKEGRTFSLVYHVDDEHSALLKEAYGEFISENGLSPLAFPSLRRMESDVISMAAELFHGNEDVAGTMTTGGTESIMMAVKAARQWAREEKGIGRPEMIVPLSVHPAFEKAAHYFDVDIQHAALGADFRVDVREVERLITPRTALIVGSAPPYPQGVLDPISELAALAQARGLLCHVDACLGGFFLPFARKLGRDIPPFDFEVPGVTSLSADLHKYGYAAKGASVVLYRNRALRRHQFFTYGGWPGGLYASPSMTGTRPGGAIAAAWAVMQALGEEGYLENARRVLSATDTLVAGINAIPGLRVLGAPQVGVFAFSSDSLNVYELGDAMEARGWKMDRQQNPPALHCMITPSHERIVEPLLADLRDCASKLAAGEPAPEGSAAMYGMVGAIPDSKQVDGFLLEFLDGVFDRT

SEQ ID NO: 23 - protein, 506 aa, Stigmatella aurantiaca
MELPNLDLLTHVPQRWLEVAEKYLKAIPQVRARLDKETGAVLSHFEKRLKPYRGTLPAFERLPAQGRPREEVLRELEGLERREETRWKEGHVSGAVYHGAEDHIAFVNEVYALHSQSNPLHAELWPSATKFEAEVVAMTANMLGATEANAGLPPEQHICGSLSSGGTESIMLAIKTYRDWARETRGITRPEMVLPSSAHPAFDKAAHYFGVKSVRVPVGADYRADVAATRRALTRNTILLVGSAPSFPHGVIDPIAELSELARKRGLGFHTDACLGGFVLPWAKRLGYPVPPFDFQLPGVTTMSADTHKFGYAAKGTSVVLYRGLALRSHQYFTATEWPGGIYFSPTFSGSRPGGLIAAAWATLVTMGEAGYLEASRHILETAKALKEGIAAIPELHVLGDPLFVIAFGSKTLDIYKVMERMGARGWDLNGLHKPPAVHLCVTLRHTQPGVAERFLEDLRAAVAHVQANPGEKGTMAPVYGMAGTVPFRGVLSDLLKKYMDLLYKV

SEQ ID NO: 24 - protein, 518 aa, Rhodococcus erythropolis
MSSTRAEELQGKIVESANLEKRAADILSRLRSLREQDAPTHGGRVLSYVYDSGLAALDELAADAARLVQPVNGLDPTVFPSTAVMERELVAFAREFLHGASDPAADTDAGGVVVGNVTSGGTESCVLAVKAARDFRGLPPGEGSLVLPTTAHAAFHKAAHLLGLELILVPVDPTSGRVGAQDIATAVRKDTVLIVVSAPSYPFAALDPIADVAEVARAHSIPLHVDACIGGLALPWWGDLPPWDFRVPGVTSMSVDFHKYGYSPKGVSVLLHRGRDLHRKQYFGLTDWPGYPVVNPTLLGSKSVAALAGAWAISQVLGQSGYAELVSRAQRATRALIGTVEAIEGLRVVGSPVGPLFAVATDDSVPLERRVDPHRWVSAVGARGFVLQGQPASTQSDGTTLPRTTHLTVTPVTESVLGELTSALVLGADDVRGSAAAEAPPALAELAGAFERGDVTVADVLALPSDAVAAALTSAGLDPRGSSDSPLDMAAVLAAVESLPREVTKRLLVEFLAGLVEP

SEQ ID NO: 25 - protein, 509 aa, Plesiocystis pacifica
MVSITAKAKQRVVDTGESLVSTLFEYAKKVPFVRDRIAAEYEKMMKDIAGQVRPYAADYPTYTELPEQGRDREAILEEMRNFHGLEADRWKDGYVSGAVYHGDREHTSFLDQAYAIHSQTNPLHSDLWPSVTKYEAEIVAMTADMLGAKKAGSGPENEVCGAVSSGGTESILLAMKTYRDWAKAEKGITSPNMVIPNTAHAAFYKAAEFFDIQLRKVTVGADCRADVKGMKRAMDRNTICLVGSAPAFPHGLVDPIEELAELAQKRKVGMHTDACLGGFVLPWARELGYPVPPFDFAVPGVTSMSADTHKYGYAAKGTSVVLYRSKALRRHQWYTVTDWPGGIYFSPTFAGSRPGGLSAACWASMQAMGREGYLDATKRIVETGQKIRRGIDAILGLKVLGDPLWVVSFASGEEGLNIYEVLDAMGKKHWNLNGLHRPPCVHLAVTLRHTKEGVAERFLDDLREAVEHTRANPGEGSGMAPIYGLAGTIPARGAVSDLLEMYLDAVYDL

SEQ ID NO: 26 - protein, 597 aa, Fluoribacter dumoffi
MFTFITGLLNSGLEAFDETFKDTPAHKIVFATAALYFIWQQYPTITQAYRSRNNTTLKQRLIDIAYSLGKNLPPVKSYLDKELNKDLQNTKDKLKELRAQMTLQDKIPEKKTPAIFLLKEFGIAPEECLFNFAGIKEGDEARRFTVKEGDGKDSGALYAVHPRELTELLKEVYAKSSLINPLHDKWPRIVAMQAEIIRWCQDLFGGSKEAYGLITHGGTTSIIEAMAAYVTHARAKGIKNPEIVVPETAHAAFKKAADLTGARLITVPVDPKSGAVNAHVMRKYISGNTAVIVGSAPSFMYGVNDPIPELGKVAQELGVPLHVDACLGGFLTAFLETSKTPMDFRVKGVTSISADLHKYGNCPKGTSVCLFSEDSPVLSVYAALNWSGGLYTTPGILDGSTSGARVAEIYTTLSYYGRKQYQEISRSIITMRQRLQDKVAELYKPDASGKRDIYIFGNPQWSVLGFRSDTLNPHFIANELDKRGWKLNSLQKPDGFHLCLTHVHTLVDRFEDKFIKDLQESIEAVKQYPADKKPEGNVKVYGTIGILPTQVQEVICRQYQRARLYYEATCTQLGFFSTPDRQENKMDISEEKKELTL

SEQ ID NO: 27 - protein, 531 aa, Polysphondylium pallidum
MNNHNGEHILEFIKSIDKYVISKYELKDIKSLVWKWSKWLAPSFHQKIKDDIENQVNQVITESFPPIEGLPLQLELPNEGLSDDEILRRLGLLQKSDVDTKQGKLFAYVYPTLERQEKIIVEAQNMFVHLNALNPTAFQSLRRMEVEVVQMIINLLNGGESARGTMTSGGTESLLMAIKTYRDRAFDLYNITEPEVVLPITAHPALEKAGRYFQVKLRYVPLVGDCQVDMRAFEKTINRNTILLVGSAPQYPHGLMDPIQEMGRLALKYKLPLHVDSCFGGLFLPFMEKLGFEVPPFDFRVPGVTSISADIHKFGYCTKGSSVLSFINDSYRMYQFMPYVGWPGGLFVSPSMLGTRGGGPIAAAWTSLVAHGENNYKEITARIMVTARAIREGINSIDGLRVIGNPVMCALAFVSENPEVNVHCIADLMQSNTKLGWKLERTHKPNAVHLTLMQSHVGKQDELIADLRQCYETVAKDPKQYINKGSAAMYSGIANIPLDNIADDFLKCFLAKTYSAPSRTAEQQPNFAKQN

SEQ ID NO: 28 - protein, 514 aa, Artificial Sequence
  His-tagged variant of sphingosine-1-phosphate lyase from Symbiobacterium thermophilum
MPLSAFSPPLPCDPARSHPTPEFPSSLQDYCEIRGIQSQPPARRDPTMDWLASLRSQIKPYRDRFPSHARLPRAGLPRAEILAEIAAMGAAESPAWRDGYASGAVYHGDEHHIAFLNEVYALQSQSNPLHPDLWPSTAKFEAEVVAMTAHMLGGDAAGGTVCGTVTSGGTESLLLAMKTYRDWARATKGITAPEAVVPVSAHAAFDKAAQYFGIKLVRTPLDADYRADVAAMREAITPNTVVVAGSAPGYPHGVVDPIPEIAALAAEHGIGCHVDACLGGFILPWAERLGYPVPPFDFRLEGVTSVSADTHKYGYGAKGTSVILYRRPDLLHYQYFIAADWPGGLYFSPTFAGSRPGALSATAWAAMLSLGEEGYLDATRRILQAADRLKAGVRAIPSLKILGDPLWVIAVASDELNIYQVMEEMAGRGWRLNGLHRPPAFHVALTLRHTEPGVVDRFLADLQDAVAQVRAHPEKATGMAPVYGMAAAAPPELVRQVLTGFIDLLYEVHHHHHH

SEQ ID NO: 29 - DNA, 1524 bp, Symbiobacterium thermophilum
gtgctttcggcgttttccccgcctttgccctgcgacccggcgcgttcccacccaacgccggaatttccctcctcattgcaggactattgcgaaattcgcggaattcaatcgcaaccgcctgcgaggagggaccccaccatggactggctcgcctccctgcggtcgcagatcaagccttaccgcgaccgctttccttcccacgcccgcctgccacgggccggcctgccccgcgccgagatcctcgccgagatcgcggccatgggcgcggccgagtccccggcctggcgggacggctatgcctccggcgccgtctaccacggcgatgagcaccacatcgcgttcctcaacgaggtctacgccctccagtcccagtccaacccgctccacccggacctctggccgagcacggccaagttcgaggccgaggtcgtcgccatgaccgcccacatgctgggcggggacgccgccggcgggaccgtctgcggcaccgtgacctccggcggcacggagtcgctcctcctggccatgaagacctaccgggactgggcccgcgcgaccaaggggatcaccgccccggaggccgtggtgccggtgagcgcgcacgccgccttcgacaaggccgcgcagtacttcggcatcaagctggtccgcaccccgctggacgccgattaccgggcagacgtcgcggccatgcgggaagccattacccccaacaccgtggtcgtcgccggatccgcgccgggctacccccacggcgtcgtcgaccccattcccgagatcgccgccctggccgcggaacacggcatcggctgccatgtggacgcctgcctgggcgggttcatcctgccgtgggcggagcggctgggctacccggtgccccccttcgatttccggctggagggcgtcacctccgtctcggccgacacccacaagtacggctacggggccaagggaacgtcggtgatcctctaccgcagacctgatctgctgcactaccagtacttcatcgcggccgactggcccggaggcctctacttctcccccaccttcgccggcagccggcccggcgcgctcagcgcgaccgcgtgggccgccatgctctccctgggggaggagggctacctggatgccacccgccggatcctgcaagccgccgaccggcttaaggcgggcgtgcgcgcgatccccagcctgaagatcctgggcgacccgctgtgggtgatcgccgtggcctcggacgagctcaacatctaccaggtgatggaggagatggccgggcggggctggcggctgaacggactgcaccggccgccggcgtttcacgtcgccctgacgcttcgccacaccgagcccggggtggtcgaccggttcctcgccgacctgcaggacgcggtggctcaggtccgggcccaccccgaaaaggcgaccgggatggccccggtctacggcatggccgccgccgcgccgccggagctggtccggcaggtgctcaccggcttcatcgacctgctgtacgaggtgtga

SEQ ID NO: 30 - DNA, 1548 bp, Artificial Sequence
  Coding sequence for his-tagged variant of sphingosine-1-lyase from Symbiobacterium thermophilum
atgccactttcggcgttttccccgcctttgccctgcgacccggcgcgttcccacccaacgccggaatttccctcctcattgcaggactattgcgaaattcgcggaattcaatcgcaaccgcctgcgaggagggaccccaccatggactggctcgcctccctgcggtcgcagatcaagccttaccgcgaccgctttccttcccacgcccgcctgccacgggccggcctgccccgcgccgagatcctcgccgagatcgcggccatgggcgcggccgagtccccggcctggcgggacggctatgcctccggcgccgtctaccacggcgatgagcaccacatcgcgttcctcaacgaggtctacgccctccagtcccagtccaacccgctccacccggacctctggccgagcacggccaagttcgaggccgaggtcgtcgccatgaccgcccacatgctgggcggggacgccgccggcgggaccgtctgcggcaccgtgacctccggcggcacggagtcgctcctcctggccatgaagacctaccgggactgggcccgcgcgaccaaggggatcaccgccccggaggccgtggtgccggtgagcgcgcacgccgccttcgacaaggccgcgcagtacttcggcatcaagctggtccgcaccccgctggacgccgattaccgggcagacgtcgcggccatgcgggaagccattacccccaacaccgtggtcgtcgccggatccgcgccgggctacccccacggcgtcgtcgaccccattcccgagatcgccgccctggccgcggaacacggcatcggctgccatgtggacgcctgcctgggcgggttcatcctgccgtgggcggagcggctgggctacccggtgccccccttcgatttccggctggagggcgtcacctccgtctcggccgacacccacaagtacggctacggggccaagggaacgtcggtgatcctctaccgcagacctgatctgctgcactaccagtacttcatcgcggccgactggcccggaggcctctacttctcccccaccttcgccggcagccggcccggcgcgctcagcgcgaccgcgtgggccgccatgctctccctgggggaggagggctacctggatgccacccgccggatcctgcaagccgccgaccggcttaaggcgggcgtgcgcgcgatccccagcctgaagatcctgggcgacccgctgtgggtgatcgccgtggcctcggacgagctcaacatctaccaggtgatggaggagatggccgggcggggctggcggctgaacggactgcaccggccgccggcgtttcacgtcgccctgacgcttcgccacaccgagcccggggtggtcgaccggttcctcgccgacctgcaggacgcggtggctcaggtccgggcccaccccgaaaaggcgaccgggatggccccggtctacggcatggccgccgccgcgccgccggagctggtccggcaggtgctcaccggcttcatcgacctgctgtacgaggtgcatcaccatcaccatcactagtaa

SEQ ID NO: 31 - protein, 605 aa, Legionella pneumophila
MFGFISDLLTAAVSSLDELLQDTPAHQIILGTAALYFLYNQYHNPSISRWCRSRNNASMKQRIIDSAYALAKNLPGVNQIIEKELNKELSSTREKLRIQRSGMTLREEIPEEGLSPQDILSAFDVDVEKCHFDFLSVTNDSPEREFLVGRGDGKDSGALYAIHPKELTELLKEVYGATALTNPLHDKWPRINAMQAEVIRWCQNLFHGSKEGYGLLTHGGTTSIIEAMAAYVIRARAKGIDYPEIVVPETAHAAFKKAAELTGAILITVPVDKKTGAVNPNVMSSYITRNTAVMVGSAPSFMNGIHDPISELGQLAKKKNVPFHVDACLGGFLTAFLDTSSEPMDFRVPGVTSISADLHKYGCCPKGTSVCLFSEDSPALSVYAALNWSGGLYATPGILDGSTSGARVAEVYATLSYYGKNKYQEIAKSIIRLRNAIQKELTALVEEGNGLTSEDIYVYGNPQWSILGFRSNTCNAHFIADELEKRGWKLNLLQNPDGFHLCLTHVHTLVGSFETQFIKDLREAVIDVKNYPPGKKPSGNVKVYGAVGMMPVELQKEICKQYQKARLDFTAASHGSLGIFAPSSTEEDDGLRNRKVGEQKVQTSL

SEQ ID NO: 32 - protein, 601 aa, Legionella pneumophila
MFGFVSDLLTAAVSSLDELLQDTPAHQIILGTAALYFLYNQYHNPSISRWYRSRNNASMKQRIIDSAYALAKNLPGVNQIIEKELNKELSSTREKLRIQRSGMTLREEIPEEGLSPQDILSAFDVDVEKCHFDFLSVTNDSPEREFLVERGDGKDSGALYAIHPKELTELLKEVYGATALTNPLHDKWPRINAMQAEVIRWCQNLFHGSKECYGLLTHGGTTSIIEAMAAYVIRARAKGIDYPEIVVPETAHAAFKKAAELTGAILITVPVDKKTGAVNPKVMSSYITRNTAVIVGSAPSFMNGIHDPVSELGQLAKKKNVPFHVDACLGGFLTAFLDTSSEPMDFRVPGVTSISADLHKYGCCPKGTSVCLFSEDSPALSVYAALNWSGGLYATPGILDGSTSGARVAEVYATLSYYGKNKYQEIAKSIITLRNAIQKELTTLLEEGNGLTSEDIYVYGNPQWSILGFRSNTCNAHFIADELEKRGWKLNLLQNPDGFHLCLTHVHTLVKGFETQFIKDLREAVIDVKNYPPGKKPSGNVKVYGAVGMMPIELQREICKQYQKARLNYTSASPGSLRIFTNVPVEEDEGLRNRKTEKYKV

SEQ ID NO: 33 - protein, 605 aa, Legionella pneumophila
MFGFISDLLTAAVSSLDELLQDTPAHQIILGTAALYFLYNQYHNPSISRWYLSRNNASMKQRIIDSAYALAKNLPGVNQIIEKELNKELSSTREKLRIQRSGMTLREEIPEEGLSPQDILSAFDVDVEKCHFDFLSVNNDSPEREFLVGGGDGKDSGALYAIHPKELTELLKEVYGATALTNPLHDKWPRINAMQAEVIRWCQNLFHGSKEGYGLLTHGGTTSIIEAMAAYVIRARAKGIDYPEIVVPETAHAAFKKAAELTGAILITVPVDKKTGAVNPRVMSSYITRNTAVIVGSAPSFMNGIHDPVSELGQLAKKKNVPFHVDACLGGFLTAFLDTSSEPMDFRVPGVTSISADLHKYGCCPKGTSVCLFSEDSPALSVYAALNWSGGLYATPGILDGSTSGARVAEVYATLSYYGKNKYQEIAKSIIKLRNAIQKELTALVEEGNGLTSEDIYVYGNPQWSILGFRSNTCNAHFIADELEKRGWKLNLLQNPDGFHLCLTHVHTLVRGFETQFIKDLREAVIDVKNYPPGKKASGNVKVYGAVGMMPIELQKEICKQYQKARLDFTAASHGSLGIFTTSPTEEDDGLRNRKVGEQKVQTSL

SEQ ID NO: 34 - protein, 600 aa, Legionella jamestowniensis
MFGAILDLLTSAVETFDETLQDTPAHTLVLATTALYFLYNQYHNPWIARAFRARHNETPKQRIIDAAYGMVKNFPLVQKYLNNELDKNLQSTRDKLSSQRAHMTLLNNIPETSRTPAEILSQFGIDLKECDFDFQSIREKDRKFIIQQGDGQDSGALYTTHPKELVEILKEVYAKTELTNPMHDKWPRINAMQAEIIRWCQNLFHGSDEGYGLLTHGGTTSIIEAMAAYVLHAKAKGIMHPEIVVPETAHAAFKKAAELTGAILITVPVDKKTGAVTVETMKKYLSHNTAVMVGSAPSFMNGINDPIGELGQLAKTRNIPLHVDACLGGFLTAFLDTSTAPMDFRVAGVSSISADTHKYGFCPKGSSVCLFSKDSPALAVYAALNWCGGLYATPGILDGSTSGARVGEIYATLSYYGRQNYQKIAESIVKLRQNLQSKSRWLSYTTTNIQPKDIYVYGNPKWSVLGFRSDTLNPHLIADELEKRGWKLNLLQNPAGFHLCLTHVHTLAEGFEDKFISDLGDAVIAVKQYPADKKPSGNVKVYGAVGTMPTDVQRQVSVQYQKERLMYTGVSNDSFFGKSTIESDIKEKKTHQPVLRYSNS

SEQ ID NO: 35 - protein, 410 aa, Marine gamma proteobacterium
MKMPDKGSSWDNLAAEMINRGSGDAKWREGKTAVYVFNAGEDVARVQKEAYALYMSENGLGPTAFPSLRKMEAEVIDMGLGLLHGSELSDGSITSGGTDSITMAVKAARDYARNALGKTGLFNIVAPYSAHPAFDKAAIMMDLELRRIPVGDDLLADCAAMSTAIDDQTIMLVGSAPNFPYGLIDPITELGEIAEAKNVWLHVDACVGGYIAPFVRMNGVDIPDFDFSVPGVSSMSADLHKYGFCAKGASTVLFKTAELKKHMIFDCRDWPGGRMLTPTLAGTRPGGAISAAWAVMNYLGIEGYKNKHQQVTDARKAIELGVRELGFTILGTPLLGIIAFQHPDVDVFAIYKQMYDRGWVTSLTTQPKALHLMLSPFHKTVTNLYLSDLTESIEQVKSGDKRAVKEAKYN

SEQ ID NO: 36 - protein, 450 aa, Artificial Sequence
  Sphingosine-1-phosphate lyase from Symbiobacterium thermophilum lacking amino acid resdidues 1 to 57 of the wildtype sequence deltaNT-FLEX deletion mutant
KPYRDRFPSHARLPRAGLPRAEILAEIAAMGAAESPAWRDGYASGAVYHGDEHHIAFLNEVYALQSQSNPLHPDLWPSTAKFEAEVVAMTAHMLGGDAAGGTVCGTVTSGGTESLLLAMKTYRDWARATKGITAPEAVVPVSAHAAFDKAAQYFGIKLVRTPLDADYRADVAAMREAITPNTVVVAGSAPGYPHGVVDPIPEIAALAAEHGIGCHVDACLGGFILPWAERLGYPVPPFDFRLEGVTSVSADTHKYGYGAKGTSVILYRRPDLLHYQYFIAADWPGGLYFSPTFAGSRPGALSATAWAAMLSLGEEGYLDATRRILQAADRLKAGVRAIPSLKILGDPLWVIAVASDELNIYQVMEEMAGRGWRLNGLHRPPAFHVALTLRHTEPGVVDRFLADLQDAVAQVRAHPEKATGMAPVYGMAAAAPPELVRQVLTGFIDLLYEV

SEQ ID NO: 37 - protein, 456 aa, Artificial Sequence
  deltaNT-FLEX deletion mutant plus C-terminal his-tag
KPYRDRFPSHARLPRAGLPRAEILAEIAAMGAAESPAWRDGYASGAVYHGDEHHIAFLNEVYALQSQSNPLHPDLWPSTAKFEAEVVAMTAHMLGGDAAGGTVCGTVTSGGTESLLLAMKTYRDWARATKGITAPEAVVPVSAHAAFDKAAQYFGIKLVRTPLDADYRADVAAMREAITPNTVVVAGSAPGYPHGVVDPIPEIAALAAEHGIGCHVDACLGGFILPWAERLGYPVPPFDFRLEGVTSVSADTHKYGYGAKGTSVILYRRPDLLHYQYFIAADWPGGLYFSPTFAGSRPGALSATAWAAMLSLGEEGYLDATRRILQAADRLKAGVRAIPSLKILGDPLWVIAVASDELNIYQVMEEMAGRGWRLNGLHRPPAFHVALTLRHTEPGVVDRFLADLQDAVAQVRAHPEKATGMAPVYGMAAAAPPELVRQVLTGFIDLLYEVHHHHHH

SEQ ID NO: 38 - protein, 458 aa, Artificial Sequence
  variant of deltaNT-FLEX deletion mutant plus C-terminal his-tag
MPKPYRDRFPSHARLPRAGLPRAEILAEIAAMGAAESPAWRDGYASGAVYHGDEHHIAFLNEVYALQSQSNPLHPDLWPSTAKFEAEVVAMTAHMLGGDAAGGTVCGTVTSGGTESLLLAMKTYRDWARATKGITAPEAVVPVSAHAAFDKAAQYFGIKLVRTPLDADYRADVAAMREAITPNTVVVAGSAPGYPHGVVDPIPEIAALAAEHGIGCHVDACLGGFILPWAERLGYPVPPFDFRLEGVTSVSADTHKYGYGAKGTSVILYRRPDLLHYQYFIAADWPGGLYFSPTFAGSRPGALSATAWAAMLSLGEEGYLDATRRILQAADRLKAGVRAIPSLKILGDPLWVIAVASDELNIYQVMEEMAGRGWRLNGLHRPPAFHVALTLRHTEPGVVDRFLADLQDAVAQVRAHPEKATGMAPVYGMAAAAPPELVRQVLTGFIDLLYEVHHHHHH